(19) United States
(12) Patent Application Publication
(10) Pub. No.: US 2010/0227841 A1
(43) Pub. Date: Sep. 9, 2010

(54) Patient populations and treatment methods

(75) Inventors: Dwight Stickney, Granite Bay, CA (US); Christopher L. Reading, San Diego, CA (US); James M. Frincke, San Diego, CA (US)

Correspondence Address: HARBOR BIOSCIENCES, INC., 4435 EASTGATE MALL, SUITE 400, SAN DIEGO, CA, 92121, US

(73) Assignee: Harbor BioSciencs, Inc., San Diego, CA (US)

(21) Appl. No.: 12/732,164

(22) Filed: Mar. 25, 2010

Related U.S. Application Data
(63) Continuation-in-part of application No. 12/731,692, filed on Mar. 25, 2010.
Application No. 12/731,692 is a continuation-in-part of application No. 12/566,565, filed on Sep. 24, 2009.
(60) Provisional application No. 61/099,880, filed on Sep. 24, 2008.
(60) Provisional application No. 61/100,246, filed on Sep. 25, 2008.
(60) Provisional application No. 61/162,620, filed on Mar. 23, 2009.
(60) Provisional application No. 61/184,283, filed on Jun. 4, 2009.
(60) Provisional application No. 61/186,360, filed on Jun. 11, 2009.

(51) Int. Cl. A61K 31/569; A61P 3/10; A61P 9/00; A61P 1/16; A61P 27/02; A61P 3/06

(52) U.S. Cl. 514/182

(57) ABSTRACT

The invention provides 17α-ethynylandrost-5-ene-3β,7β,17β-triol for use in methods to treat, e.g., pre-diabetic hyperglycemia or type 2 diabetes patients having a fasting blood glucose level of at least 110 mg/dL or a post prandial glucose level of at least about 140 mg/dL an optionally a body mass index of >29.9, wherein the patients are not concurrently treated with metformin and optionally another antidiabetic agent. In these methods, the patient will preferably have a fasting insulin level of at least 3.5 to 4 μU/mL.

### CROSS REFERENCE TO RELATED APPLICATIONS

This application claims priority to and is a continuation-in-part of pending U.S. application Ser. No. 12/731,692, filed Mar. 25, 2010, which claims priority to and is a continuation-in-part of pending U.S. application Ser. No. 12/566,565, filed Sep. 24, 2009, filed Sep. 24, 2009, both of which are incorporated herein by reference in their entireties. This application claims priority to abandoned provisional application Ser. No. 61/099,880, filed Sep. 24, 2008, abandoned provisional application Ser. No. 61/100,246, filed Sep. 25, 2008, abandoned provisional application Ser. No. 61/162,620, filed Mar. 23, 2009, pending provisional application Ser. No. 61/184,283, filed Jun. 4, 2009 and pending provisional application Ser. No. 61/186,360, filed Jun. 11, 2009, all of which are incorporated herein by reference in their entireties.

### FIELD OF THE INVENTION

The invention relates to a screening method to treat patient subpopulations that are enriched for treatment responders in treating metabolic or cardiovascular disease conditions or symptoms, e.g., obesity, type 1 diabetes, type 2 diabetes, hyperglycemia, hepatitis or hypercholesterolemia.

### BACKGROUND

Many metabolic diseases and related conditions such as type 2 diabetes, prediabetes (insulin resistance) and obesity are typically characterized by a range of symptoms. Individual patients can vary in the symptoms that they display and in the rate at which a condition such as obesity may progress to overt diabetes or in the rate at which a diagnosed type 2 diabetic patient progresses to develop neuropathy, retinopathy or other diabetes associated conditions. Individual patient phenotypes for biomolecules vary widely, which complicates assessment of individuals with regard to their true clinical status.

A range of drugs and treatment protocols have been developed to treat metabolic diseases and the drugs include metformin, exenatide, nateglinide, valsartan, sulfonylureas such as glyburide and glipizide and thiazolidinediones such as rosiglitazone maleate and pioglitazone HCl. American Diabetes Association position statement, Diabetes Care, 31 supp 1:S12-S54, 2008.

One method to assess the clinical status of individual pre-diabetic and diabetic patients is to perform a glucose clamp assay (euglycemic hyperinsulinemic clamp technique), which measures whole body glucose metabolism. The rate at which glucose is utilized in patients in this assay, the M value, measures the rate at which peripheral tissues, primarily muscle, take glucose up from the blood. A higher rate of uptake (higher M value) indicates a more normal glucose metabolism in the individual patient. Lower M values generally indicate impaired glucose metabolism and can correlate with prediabetes and overt diabetes. Methods to perform glucose clamp studies have been described. See, e.g., A. Brehm and M. Roden, Glucose Clamp Techniques, in Clinical Diabetes Research: Methods and Techniques, M. Roden editor, chapter 4, pages 43-76, 2007, John Wiley & Sons, Ltd. Important drawbacks with performing the glucose clamp assay are that the procedure takes several hours to perform, is expensive and is very labor intensive. During the assay, patients must have an indwelling catheter inserted in a vein and the protocol requires heating one hand for the length of the procedure. These drawbacks prevent large scale use of the clamp protocol to identify patient subpopulations in most clinical trials that assess treatments for dysregulated glucose metabolism conditions, e.g., type 2 diabetes or hyperglycemia.

Another method to assess the clinical status of individual pre-diabetic and diabetic patients is to rely on homeostatic model assessment (HOMA) to assess glucose, insulin or pancreatic islet β-cell function. T. M. Wallace et al, Diabetes Care, 27(6):1487-1495, 2004. HOMA clinical assessments for patients lead to variable outcomes for treatment protocols, so this approach has not been uniformly successful in identifying patient populations that will respond to therapy for glucose dysregulation, e.g., type 2 diabetes or hyperglycemia.

Methods and means to detect biomolecules such as leptin, retinol binding protein 4 (RBP4), and C-reactive protein (CRP) in various samples (cells) and biological fluids (blood, serum or plasma) have been described (e.g., U.S. Pat. Nos. 6,867,005, 5,624,597, 5,358,852).

Biomolecules such as leptin, RBP4, MCP1 and CRP have not yet been approved by any regulatory agency as a surrogate or end point for treating any disease.

To date, no surrogate biomarker has been identified that mimics the pathological status of patients having a low M value, typically equal to or less than about 5 mg/kg/min, typically less than about 5.0±0.3. Patients having a low M value are enriched for patients that better respond to treatments for glucose dysregulation conditions such as diabetes, prediabetes, hyperglycemia and sometimes obesity itself with or without accompanying prediabetes or diabetes.

### DESCRIPTION OF THE INVENTION

Summary. In one embodiment, the invention provides a treatment method for a patient(s) having pre-diabetic hyperglycemia or type 2 diabetes, comprising, (a) determining the body mass index (BMI) of the patient(s); (b) in a patient(s) of step (a) that is obese (having a BMI of >29.9, e.g., 30.0), determining the fasting blood or serum glucose level and/or the post prandial glucose level after an oral 75 g glucose load; (c) selecting patient(s) with a fasting blood glucose level of at least 110 mg/dL or a post prandial glucose level of at least about 140 mg/dL as patient(s) for treatment; and (d) treating the patient(s) of step (c) with an effective amount of 17α-ethynylandrost-5-ene-3β,7β,17β-triol, wherein the patient(s) have and wherein the patient(s) has not been treated with metformin and optionally wherein the patient(s) has not been treated with another insulin sensitizer or another antidiabetic agent.

Other embodiments are described elsewhere herein.

Detailed description. As used herein and unless otherwise stated or implied by context, terms that are used herein have the meanings defined below or elsewhere herein. Unless otherwise contraindicated or implied, e.g., by including mutually exclusive elements or options, in these definitions and anywhere the specification, claims or elsewhere herein, the terms “a” and “an” mean one or more than one. The term “or” means and/or.

As used herein, a subject is typically a mammal, preferably one that can be used in studies of metabolic disorders and preferably a human, typically a human having a metabolic disorder such as diabetes or hyperglycemia. Non-human subjects include dogs and rodents such as mice and rats. Subjects can exclude rodents such as mice or rats.

Drug or drugs refer to compounds that have been approved for one or more human clinical uses and a drug candidate is a compound that can potentially be developed or approved for human clinical use.

Methods and means to detect biomolecules such as leptin, retinol binding protein 4 (RBP4), C-reactive protein (CRP), insulin and monocyte chemoattractant protein 1 (MCP1) in various samples and biological fluids have been described (e.g., U.S. Pat. Nos. 6,867,005, 5,624,597, 5,358,852 and J. A. de Lemos et al., Circulation, 107:690-695, 2003, I. Sylvester et al., J. Immunology, 151(6):3292-3298, 1993).

The present invention provides methods to assess the pathological status of obese, hyperglycemic, pre-diabetic and diabetic subjects or human patients using baseline levels of one, two or more of the specific biomarkers, RBP4, CRP, leptin, MCP1, TNFα, resistin, IL-6 or IL-1β, preferably one or two of MCP1, RBP4, CRP and leptin as surrogates in lieu of performing the glucose clamp protocol. Also preferred is the use of MCP1 in lieu of performing the glucose clamp protocol, optionally in combination with patients, e.g., hyperglycemic, having a BMI of about 28 or greater, e.g., at least about 31. Also preferred is treatment of patients having a BMI of at least about 28 or, at least about 31 for treatment naïve patients, and pre-diabetic hyperglycemia, type 2 diabetes or one of the other conditions described herein, e.g., arteriosclerosis, nonalcoholic steatohepatitis or a microvascular disease (e.g., nephropathy) herein wherein the patients have a BMI of at least about 28 or at least about 31 for treatment naïve diabetes patients. The subjects or patients may be obese with or without accompanying prediabetes or diabetes. This assessment identifies subject phenotypes that are more likely to respond to treatment with agents that can partially normalize or completely normalize one or more of these surrogate biomarkers in obese or prediabetic subjects and in subjects or patients that have type 2 diabetes, hyperglycemia, insulin resistance or another disease or condition described herein. Normalization of a biomarker means that the level or activity of the molecule is within normal ranges for the human or animal and near normalization of a biomarker means that the level or activity of the molecule is preferably within about ±50% of normal ranges or more preferably within about ±20% of normal ranges for the patient or for levels in comparable normal subjects. Preferred treatments use 17α-ethynylandrost-5-ene-3β,7β,17β-triol as the drug, e.g., in dosages as described herein (e.g., about 4 mg/day to about 200 mg/day or about 10 mg/day to about 100 mg/day). Such dosages provide drug exposure levels as described herein.

In some embodiments, two or three drugs are co-administered to a patient having a body mass index of about 28-31 and a clinical condition such as type 2 diabetes or hyperglycemia and a surrogate marker profile described herein. Such co-administration protocols include a two drug combination of 17α-ethynylandrost-5-ene-3β,7β,17β-triol and an antidiabetic or anti-hyperlipidemic agent selected from the group consisting of pioglitazone, troglitazone, ciglitazone and rosiglitazone. Such co-administration protocols include a two drug combination of 17α-ethynylandrost-5-ene-3β,7β,17β-triol and an antidiabetic or anti-hyperlipidemic agent optionally selected from the group consisting of tolbutamide, chlorpropamide, tolazamide and acetohexamide. Such co-administration protocols include a drug combination of 17α-ethynylandrost-5-ene-3β,7β,17β-triol and an antidiabetic or anti-hyperlipidemic agent optionally selected from the group consisting of glyburide, glipizide, gliclazide, glimepiride, gliquidone, glibornuride, glisoxepid, glisentide, glisolamide, glybuzole, glyclopyramide and a meglitinide. Such co-administration protocols preferably include a two drug combination of 17α-ethynylandrost-5-ene-3β,7β,17β-triol and an antidiabetic or anti-hyperlipidemic agent optionally selected from the group consisting of metformin (preferred), buformin and phenformin. Such co-administration protocols also preferably include a two drug combination of 17α-ethynylandrost-5-ene-3β,7β,17β-triol and an antidiabetic or anti-hyperlipidemic agent optionally selected from the group consisting of lovastatin, fluvastatin, pravastatin, simvastatin, or atorvastatin, or a pharmaceutically suitable acid addition salt or a salt formed with a pharmaceutically suitable base thereof.

In these two drug combinations, the 17α-ethynylandrost-5-ene-3β,7β, 17β-triol is administered as described elsewhere herein, e.g., daily oral dosing with (i) 5 mg dosed once or twice per day (5 or 10 mg/day), (ii) 10 mg dosed once or twice per day (10 or 20 mg/day) or (iii) 20 mg dosed once or twice per day (20 or 40 mg/day). The other drugs, e.g., metformin, rosiglitazone, glyburide or simvastatin are dosed as normally used or at lower dosages, e.g., at about a 50% lower daily dose or about an 80% lower daily dose. For example, daily doses of simvastatin are generally 20 mg/day or 40 mg/day in normal use, but can be decreased to 10 mg/day or as low as 4 mg/day to assess the effect of the combination therapy on the surrogate markers and/or the underlying disease itself. Similarly, a daily dose of 1 g of metformin (typically administered as 500 mg twice per day) can be decreased to about 0.5 g/day or about 0.2 g/day. A daily dose of 1.5 g/day or 2 g/day of metformin (typically administered as 750 mg or 1000 mg twice per day) can be decreased to about 1 g/day or about 0.5 g/day. Dosages for other drugs have been described. American Diabetes Association position statement, Diabetes Care, 31 supp 1:S12-S54, 2008.

Identification of a responder phenotype (hyperglycemic patients having a BMI of at least about 28 who are also taking an insulin sensitizer such as metformin) and/or one or more dysregulated surrogate biomarkers described herein such as one, two, three or more of IL-1β, IL-6, MCP1, TNFα, CRP, resistin or RBP4 for patients having a M<5 whole body glucose metabolism rate (<5 mg/kg/min) identifies patients or patient populations that are more likely to respond to therapy. Preferred treatments will be with 17α-ethynylandrost-5-ene-3β,7β,17β-triol, optionally in combination therapy with another drug. This permits design of clinical trials that require smaller numbers of patients to obtain statistical significance for success or failure of a drug candidate or combination. This patient population is identified without the need to perform the glucose clamp procedure. These surrogate biomarkers for the patient's M value permits diagnosis of patients who are likely to respond to therapy and the continued monitoring of these surrogate biomarkers permits assessment of patients who may respond at one time and then relapse after some period of therapy. A relapse can indicate a significant change in a patient's clinical status and it could dictate a change in prognosis or therapy. Periodic monitoring, e.g., about every 2 months, about every 6 months or about every 12 months, of the surrogate biomarker(s) for the M value would indicate that a patient remains treatment responsive so long as the surrogate biomarker(s) remain at normal or near normal levels in the treated patient. Any of these methods can be adapted for use, e.g., as described in the claims and description herein.

All citations herein are incorporated herein by reference with specificity and any one or more of these citations are optionally appended to this paragraph.

Invention embodiments include a method to treat a responder patient having hyperglycemia or diabetes or to slow the progression of hyperglycemia or diabetes, comprising (a) identifying patients having hyperglycemia or diabetes with a body mass index of at least 28, are taking an insulin sensitizer such as metformin and a fasting plasma insulin level of ≧4 μU/mL to identify or obtain a responder patient; and (b) administering an effective amount of 17α-ethynylandrost-5-ene-3β,7β,17β-triol to the responder patient, optionally 5 mg/day, 10 mg/day or 20 mg/day. The responder patients may also be further identified in step (a) as having a serum monocyte chemoattractant protein-1 (MCP-1, or MPC1) level of about 400 pg/mL and/or a fasting C-peptide level of ≧about 4 ng/mL or preferably about 2 ng/mL. In one preferred embodiment, an effective amount of 17α-ethynylandrost-5-ene-3β,7β,17β-triol is administered at 10 mg/day, typically as two 5 mg oral doses taken orally, e.g., at 12±2 hr intervals or at about at about 12±4 hr intervals. In another more preferred embodiment, an effective amount of 17α-ethynylandrost-5-ene-3β,7β,17β-triol is administered at 50 mg/day, typically as two 25 mg oral doses taken orally at about 12 hour intervals (e.g., 12±4 hrs). Responder patients may be diagnosed as having pre-diabetic hyperglycemia or as having type 1 diabetes or type 2 diabetes. Responder patients may be diagnosed or considered as having another disease or condition described herein, e.g., a microvascular disease, a macrovascular disease or nonalcoholic steatohepatitis.

Invention embodiments include a method to treat a responder patient having hyperglycemia or diabetes or to slow the progression of hyperglycemia or diabetes, comprising (a) identifying patients having hyperglycemia or diabetes with a body mass index of at least 31, are not taking an insulin sensitizer such as metformin or another antidiabetic agent and a fasting plasma insulin level of 4 μU/mL to identify or obtain a responder patient; and (b) administering an effective amount of 17α-ethynylandrost-5-ene-3β,7β,17β-triol to the responder patient, optionally 5 mg/day, 10 mg/day or 20 mg/day. The responder patients may also be further identified in step (a) as having a serum monocyte chemoattractant protein-1 (MCP-1, or MPC1) level of about 400 pg/mL and/or a fasting C-peptide level of about 4 ng/mL or preferably about 2 ng/mL. In one preferred embodiment, an effective amount of 17α-ethynylandrost-5-ene-3β,7β,17β-triol is administered at 10 mg/day, typically as two 5 mg oral doses taken orally, e.g., at 12±2 hr intervals or at about at about 12±4 hr intervals. In another more preferred embodiment, an effective amount of 17α-ethynylandrost-5-ene-3β,7β,17β-triol is administered at 50 mg/day, typically as two 25 mg oral doses taken orally at about 12 hour intervals (e.g., 12±4 hrs). Responder patients may be diagnosed as having pre-diabetic hyperglycemia or as having type 1 diabetes or type 2 diabetes. Responder patients may be diagnosed or considered as having another disease or condition described herein, e.g., a microvascular disease, a macrovascular disease or nonalcoholic steatohepatitis.

Other preferred embodiments of the foregoing method include a treatment method wherein the responder patient is overweight or obese with a body mass index of at least 28 or at least 29 and a fasting insulin level of at least about 10 μU/mL or more preferably at least about 5 μU/mL. Such responder patients will generally be pre-diabetic, but they can also be type 1 or type 2 diabetics. Preferred body mass index values for treating adult human females is about 28 kg/m² to about 37 kg/m² and about 28 kg/m² to about 39 kg/m² for males.

Pre-diabetic hyperglycemia is generally observed as a fasting plasma glucose in the range of 100 mg/dL to 125 mg/dL, while diabetes is typically diagnosed by a fasting plasma glucose level of at least 126 mg/dL. Other indicators described herein can alternatively be used, e.g., elevated HbA1c of at least about 7% is indicative of hyperglycemia. Progression of prediabetic hyperglycemia to overt diabetes is thus observable by monitoring fasting plasma glucose levels on several occasions in a patient over time. A significant proportion of prediabetic hyperglycemic patients will be overweight or obese with a body mass index (BMI) of at least 28 or 29. Individuals having a BMI of about 25-29 are typically considered to be overweight and individuals having a BMI of about 30-35 are typically considered to be obese. Extreme obesity is defined as a BMI of >40.0. A significant proportion of such overweight or overweight or obese prediabetic hyperglycemic patients, particularly those having (i) a BMI of at least 28 and are taking an insulin sensitizer such as metformin and (ii) a BMI of at least 31 and are not taking an insulin sensitizer such as metformin, are within the responder patient subpopulations described herein and such patients can be treated according to the methods described herein.

Invention embodiments include a method to identify or treat a responder patient(s) having diabetes, hyperglycemia, macrovascular disease or hypercholesterolemia comprising, (a) measuring the body mass index (BMI) of a patient(s) having diabetes, hyperglycemia, macrovascular disease or hypercholesterolemia; (b) in a patient(s) of step (a) having a BMI of at least about 28, measuring the level of (i) MCP1 (monocyte chemoattractant protein 1) and (ii) insulin and/or C peptide; (c) recording the values of MCP1 and insulin and selecting patient(s) with (i) significantly elevated MCP1 and (ii) an insulin level of at least about 4 μU/mL and/or a C peptide level of at least about 2 ng/mL as responder patient(s); and (d) optionally treating the responder patients of step (c) with a therapeutic agent. Optimal responder patients typically have (a) a body mass index of at least about 28, e.g., at least 28, 28.5, 29 or higher and who are receiving an insulin sensitizer such as metformin and (b) a body mass index of at least about 31, e.g., at least 30.5, 31.5, 32 or higher and who are not receiving an insulin sensitizer such as metformin. In some embodiments, responder patients may also have other conditions, e.g., hypercholesterolemia (total cholesterol of at least about 220 mg/dL or at least about 240 mg/dL or as assessed by a clinician) or hyperglycemia (fasting serum glucose of at least about 110 mg/dL, usually at least about 140 mg/dL or as assessed by a clinician). In other embodiments, responder patients may also have or have experienced arteriosclerosis or a macrovascular disease or event such as arterial hypertension (typically where systolic blood pressure is at least about 140 mm Hg and/or diastolic blood pressure is at least about 90 mm Hg) a myocardial infarction or stroke.

In some embodiments, patients having a BMI of at least about 28 (e.g., >28 or 29) or BMI of at least about 31 (e.g., >30.5 or ≧32) and hypercholesterolemia, are treated, e.g., with an effective amount of 17α-ethynylandrost-5-ene-3β,7β,17β-triol and optionally a statin such as atorvastatin (10-80 mg/day), lovastatin (10-80 mg/day), simvastatin (5 to 80 mg/day) or rosuvastatin (5 to 40 mg/day).

Patients to be treated as described herein may have hyperglycemia. Hyperglycemic patients have a fasting serum glucose of at least about 100 mg/dL or at least about 110 mg/dL, usually at least about 140 mg/dL or as assessed by a clinician. Such patients may occasionally be glucose intolerant, i.e., having a fasting plasma glucose >110 and <126 mg/dL or a 2-hr post prandial glucose level of 140-199 mg/dL after 75 g oral glucose intake (OGTT). A fasting plasma glucose level of >126 mg/dL is now considered to be overtly diabetic, although the prior definition considered frank diabetes to require a fasting plasma glucose level of 140 mg/dL (Merck Manual, 7^{th} edition, 1999, page 170, Merck Research Laboratories, Whitehouse Station, N.J.). As used herein, glucose intolerance and hyperglycemia are based on current medical definitions, unless otherwise stated, e.g., a fasting plasma glucose level of >126 mg/dL is diabetic.

In hyperglycemic patients, the level of HbA1c will usually be at the high end of normal or elevated, e.g., at least 7.0%, and usually at least about 8%, preferably at least about 7.1% to about 12% or greater. Elevated glucose, as reflected by elevated, HbA1c contributes to the development and progression of microvascular disease, e.g., nephropathy. Elevated HbA1c reflects impaired glucose metabolism and contributes to the development and progression of microvascular disease including neuropathy.

The treatment methods described herein may involve treating patient subsets having elevated MCP1 (monocyte chemoattractant protein 1, or sometimes referred to as CCL2, which is CC chemokine receptor 2 ligand). CC chemokine receptor 2 (CCR2) is the receptor for MCP1. Effects of treatment with 17α-ethynylandrost-5-ene-3β,7β,17β-triol indicated a decrease in expression levels of CCR2 and a trend to decrease MCP1 in circulation (typically as measured as protein levels in serum, e.g., by ELISA assay). These effects are consistent with biological responses that correspond to reduced progression and/or at least partial reversal of cardiovascular plaque accumulation. That provides a basis for treating, e.g., slowing the progression, of arteriosclerosis and atherosclerosis. Such treatments would be effective in patients having a dyslipidemia such as hypercholesterolemia and/or hypertriglyceridemia with or without also having an arteriosclerosis condition such as atherosclerosis. Such patients will preferably have BMI and fasting plasma insulin levels as described in the embodiments described below (e.g., in embodiment 1, 9, 10, 17 or 19), examples or original claims, e.g., a BMI of ≧28 and insulin of ≧4 μU/mL. Any of these patients may also have hyperglycemia or overt diabetes. Patients with elevated MCP1 are also treatable for preventing or reducing the severity of cardiovascular disease, including myocardial infarction.

Lipidomic analysis in rats treated with 17α-ethynylandrost-5-ene-3β,7β, 17β-triol (example 10) revealed that liver cholesterol and triglyceride content was reduced. Also observed was an elevation of LDL receptor and HMG-CoA reductase expression with an accompanying decrease in total serum cholesterol. The in vivo activity of 17α-ethynylandrost-5-ene-3β,7β,17β-triol appears to include inhibition of hepatic lipogenesis. Amelioration of hepatic lipid accumulation is associated with an improvement in insulin sensitivity.

Therefore, the drug-induced reduction of hepatic triglyceride and cholesterol content may participate in the effects on hepatic insulin sensitivity. The compound is thus useful for treating hepatitis such as non-alcoholic steatohepatitis (NASH) or alcoholic hepatitis (e.g., acute alcoholic hepatitis). NASH is accompanied by insulin resistance and pathological inflammation mediated through the TLR4 pathway. Treatment with 17α-ethynylandrost-5-ene-3β,7β,17β-triol ameliorates insulin resistance and it appears to exert anti-inflammatory effects through the TLR4 pathway (see example 10 below). In addition, increased macrophage infiltration and accumulation in adipose tissue occurs in obesity and reduction in such macrophage chemotaxis would be expected to ameliorate or slow the progression of macrovascular conditions such as arteriosclerosis and atherosclerosis.

Specific embodiments of the invention and aspects thereof are described further in the following embodiments.

### Embodiment 1

A method to identify responder patient(s) having a metabolic or related disorder such as diabetes, hyperglycemia, macrovascular disease, a microvascular disease or a dyslipidemia or hyperlipidemia condition such as hypercholesterolemia or hypertriglyceridemia comprising, (a) measuring the body mass index (BMI) of a patient(s) having diabetes, hyperglycemia, macrovascular disease or hypercholesterolemia; (b) in a patient(s) of step (a) having a BMI of at least about 28, measuring the level of (i) MCP1 and (ii) insulin and/or C peptide; (c) recording the values of MCP1, insulin and/or C peptide and selecting patient(s) with (i) significantly elevated MCP1 and (ii) an insulin level of at least about 4 μU/mL and/or a C peptide level of at least about 2 ng/mL as responder patient(s); and (d) optionally treating the responder patients of step (c) with 17α-ethynylandrost-5-ene-3β,7β,17β-triol. This embodiment includes patients having a BMI of about 28 or greater and that are insulin resistant and/or inflamed, e.g., as measured by elevated MCP1, represent a metabolic or related disorder phenotype that generally responds more favorably to treatment, e.g., treatment with 17α-ethynylandrost-5-ene-3β,7β,17β-triol, than patients who do not have this phenotype. The biological response to treatment is typically observable as improved glucose control in pre-diabetic and diabetic patients who have at least some remaining β-cell function and insulin synthesis capacity. Improved glucose control is observed by a decrease in HbA1c, HOMA assessments or by other diabetes-associated diagnostic assessments. Improvement in dyslipidemia or hyperlipidemia is observed, e.g., as decreased total cholesterol in hypercholesterolemia or decreased triglyceride levels in hypertriglyceridemia. Benefit in cardiovascular or macrovascular disease is observed, e.g., as decreased CRP in treated patients. Determining sufficient (normal or near normal) β-cell function or insulin synthesis capacity is preferably determined by measuring fasting plasma insulin levels of at least about 4 μU/mL or at least about 5 μU/mL to the upper limit of normal, e.g., about 29. Treatments for these conditions will typically be prolonged or chronic, e.g., for at least about 3 months or least about 6 months, because these diseases and conditions tend to be chronic. These diseases and conditions will tend to respond to treatment gradually. In preferred embodiments, the patients are also treated with an insulin sensitizer such as metformin.

2. The method of embodiment 1 wherein the serum level of MCP1 is at least about 400 pg/mL. Patient MCP1 levels are typically about 450 pg/mL or greater. MCP1 levels in the responder patient populations can be at least about 500 pg/mL. MCP1 levels in the responder patients populations can be ≧400 pg/mL. MCP1 levels in the responder patients populations can be ≧450 pg/mL. MCP1 levels in the responder patients populations can be ≧500 pg/mL.

3. The method of embodiment 1 or 2 wherein the responder patient(s) has a BMI of at least 28.

4. The method of embodiment 1, 2 or 3 wherein the responder patient(s) has a BMI of at least about 28.5. The method of embodiment 1, 2 or 3 wherein the responder patient(s) has a BMI of at least about 29. The method of embodiment 1, 2 or 3 wherein the responder patient(s) has a BMI of at least about 30. The method of embodiment 1, 2 or 3 wherein the responder patient(s) has a BMI of ≧28. The method of embodiment 1, 2 or 3 wherein the responder patient(s) has a BMI of ≧28.5. The method of embodiment 1, 2 or 3 wherein the responder patient(s) has a BMI of ≧29.

5. The method of embodiment 1, 2, 3 or 4 wherein the responder patient(s) has a fasting plasma insulin level of at least 4 μU/mL and/or a C peptide level of at least 2.5 ng/mL. The method of embodiment 1, 2, 3 or 4 wherein the responder patient(s) has a fasting plasma insulin level of ≧4.5 μU/mL.

6. The method of embodiment 1, 2, 3 or 4 wherein the responder patient(s) has a fasting plasma insulin level of at least about 5 μU/mL and/or a C peptide level of at least about 3 ng/mL. The method of embodiment 1, 2, 3 or 4 wherein the responder patient(s) has a fasting plasma insulin level of ≧5 μU/mL.

7. The method of embodiment 1, 2, 3, 4, 5 or 6 wherein the responder patient(s) has type 2 diabetes.

8. The method of embodiment 1, 2, 3, 4, 5 or 6 wherein the responder patient(s) has type 1 diabetes.

9. The method of embodiment 1, 2, 3, 4, 5 or 6 wherein the responder patient(s) has hyperglycemia.

10. The method of embodiment 1, 2, 3, 4, 5 or 6 wherein the responder patient(s) has hyperglycemia, but not diabetes, e.g., pre-diabetic hyperglycemia or hyperglycemia associated with a trauma such as a stroke or myocardial infarction.

11. The method of embodiment 1, 2, 3, 4, 5 or 6 wherein the responder patient(s) has hypercholesterolemia.

12. The method of embodiment 1, 2, 3, 4, 5 or 6 wherein the responder patient(s) has, or has experienced, macrovascular disease, optionally one or more of arteriosclerosis, atherosclerosis, hypertension, thromboembolism, myocardial infarction or stroke. In some of these embodiments, the patients will have arteriosclerosis. In some of these embodiments, the patients will have hypertension. In some of these embodiments, the patients will have experienced a myocardial infarction or stroke, preferably at about a time within the preceding 24 to 96 hours, e.g., preferably within about 48 hours of the event or within about 24-36 hours of the event.

13. The method of embodiment 7, 8, 9, 10 or 11 wherein the responder patient(s) has a BMI of at least about 28.5. The method of embodiment 7, 8, 9, 10 or 11 wherein the responder patient(s) has a BMI of at least about 29. The method of embodiment 7, 8, 9, 10 or 11 wherein the responder patient(s) has a BMI of 29. The method of embodiment 7, 8, 9, 10 or 11 wherein the responder patient(s) has a BMI of at least about ≧30. The method of embodiment 7, 8, 9, 10 or 11 wherein the responder patient(s) has a BMI of 30. In some of these embodiments, the patients will have hyperglycemia. In some of these embodiments, the patients will have type 2 diabetes. In some of these embodiments, the patients will have hypercholesterolemia.

14. The method of embodiment 13 wherein the responder patient(s) has an insulin level of at least about 4 μU/mL. The method of embodiment 13 wherein the responder patient(s) has an insulin level of 4 μU/mL.

15. The method of embodiment 13 wherein the responder patient(s) has an insulin level of at least about 4.5 μU/mL. The method of embodiment 13 wherein the responder patient(s) has an insulin level of 4.5 μU/mL.

16. The method of embodiment 13 wherein the responder patient(s) has an insulin level of at least about 5 μU/mL. The method of embodiment 13 wherein the responder patient(s) has an insulin level of 5 μU/mL.

17. Use of an compound for the preparation of a medicament (a) for the treatment of diabetes, hyperglycemia, a macrovascular disease, a microvascular disease, nonalcoholic steatohepatitis or hyperlipidemia in a patient(s) having (i) a BMI of at least about 28, (ii) optionally has a significantly elevated MCP1 and/or (iii) optionally is insulin resistant or has an insulin level of at least about 4 μU/mL and/or a C peptide level of at least about 2 ng/mL, wherein the compound is 17α-ethynylandrost-5-ene-3β,7β,17β-triol. Hyperlipidemia includes hypertriglyceridemia and hypercholesterolemia. The patient(s) that have a fasting blood insulin level of at least about 4 μU/mL will usually have a fasting blood insulin level of at about 4 μU/mL to about 29 μU/mL, which is the normal range for insulin. Treatments for these conditions will typically be prolonged or chronic, e.g., for at least about 3 months or least about 6 months, because these diseases and conditions tend to be chronic. These diseases and conditions will tend to respond to treatment gradually. In some embodiments the patients have diabetes and are also taking an insulin sensitizer such as metformin. In other embodiments the patients have a BMI of at least 31 (≧30.5 or ≧32) and have diabetes but are not taking metformin or optionally another an insulin sensitizer such as rosiglitazone.

18. Use according to embodiment 17 wherein the subject(s) or patient(s) has a BMI of at least 28 or at least about 28.5 or at least about 29 or at least about 30. Specific uses include diabetes patients having a BMI of at least 28. Other embodiments include uses where the patient has a BMI of at least 29 and has diabetes. Other specific embodiments also include uses the patient has a BMI of at least 28, e.g., about 29 or above, and has hyperglycemia. Other specific embodiments also include uses where the patient has a BMI of at least 28, e.g., about 29 or above, and has hypertriglyceridemia. Other specific embodiments also include uses where the patient has a BMI of at least 28, e.g., about 29 or above, and has hypercholesterolemia. In any of these specific embodiments, the patient(s) may be insulin resistant. In any of these specific embodiments, the patient(s) may be insulin resistant and have an elevated level of MCP-1, e.g., at least 400 pg/mL. The elevated MCP-1 may be determined in circulation or in cells or tissue such as white adipose tissue or macrophages, which may be in liver or white adipose tissue. That may be measured by PCR methods to determine MPC1 RNA in cells ex vivo. However, MCP-1 is usually measured in blood or plasma or blood serum, preferably plasma. The elevated MCP-1 in circulation in any of these specific embodiments may be at least 450 pg/mL. The elevated MCP-1 in circulation in any of these specific embodiments may be at least 500 pg/mL, e.g., about 500-2,500 pg/mL. The patient(s) in any of these specific embodiments may have type 2 diabetes, e.g., type 2 diabetes, a BMI of at least 28 or at least 29 and are taking metformin or another insulin sensitizer. The patient(s) in any of these specific embodiments may have type 2 diabetes, e.g., type 2 diabetes, a BMI of at least 31 or at least 32, but are not taking metformin or another insulin sensitizer.

19. Use according to embodiment 17 or 18 wherein the subject(s) or patient(s) has a serum MCP1 level of at least about 400 pg/mL.

20. Use according to embodiment 17, 18 or 19 wherein the subject(s) or patient(s) has an insulin level of at least 4 μU/mL or at least about 4.5 μU/mL or at least about 5 μU/mL or at least about 6 μU/mL.

21. Use according to embodiment 17, 18, 19 or 20 wherein the subject(s) or patient(s) has type 2 diabetes.

22. Use according to embodiment 17, 18, 19 or 20 wherein the subject(s) or patient(s) has type 1 diabetes.

23. Use according to embodiment 17, 18, 19 or 20 wherein the subject(s) or patient(s) has hyperglycemia.

24. Use according to embodiment 21, 22 or 23 wherein the subject(s) or patient(s) has, or has experienced, a macrovascular disease or event, optionally one or more of arteriosclerosis, atherosclerosis, thromboembolism, myocardial infarction or stroke. Specific embodiments include uses where the subject(s) or patient(s) has arteriosclerosis. Other specific embodiments include uses where the subject(s) or patient(s) has atherosclerosis. Specific embodiments also include uses where the subject(s) or patient(s) has experienced a thromboembolism, myocardial infarction or stroke.

25. A method to treat a patient having hyperglycemia or diabetes, comprising (a) identifying patients having hyperglycemia or diabetes with a body mass index of at least about 28, and one, two or three of (i) a fasting plasma insulin level of ≧4 μU/mL, (ii) a serum monocyte chemotactic protein-1 (MCP-1) level of ≧400 pg/mL and (iii) a fasting C-peptide level of 2 ng/mL to identify a responder patient; and (b) administering an effective amount of 17α-ethynylandrost-5-ene-3β,7β,17β-triol to the responder patient, optionally 5 mg/day, 10 mg/day or 20 mg/day. The patient(s) that have a fasting blood insulin level of at least about 4 μU/mL will usually have a fasting blood insulin level of at about 4 μU/mL to about 29 μU/mL, which is the normal range for insulin. Treatments for these conditions will typically be prolonged or chronic, e.g., for at least about 3 months or least about 6 months, because these diseases and conditions tend to be chronic. These diseases and conditions will tend to respond to treatment gradually.

26. The method of embodiment 25 wherein the responder patients have a body mass index of at least about 28, a fasting plasma insulin level of 4 μU/mL and a MCP-1 level of 400 pg/mL.

27. The method of embodiment 25 wherein the responder patients have a body mass index of at least about 29 and/or a fasting plasma insulin level of 6 μU/mL.

28. The method of embodiment 25 wherein the responder patients have a body mass index of at least 28 and a fasting plasma insulin level of ≧4 μU/mL, a MCP-1 level of ≧400 pg/mL and fasting C-peptide level of ≧2 ng/mL.

29. The method of embodiment 25 wherein the responder patients have a body mass index of at least 29 and a fasting plasma insulin level of ≧4 μU/mL, a MCP-1 level of ≧400 pg/mL and fasting C-peptide level of ≧2 ng/mL.

30. The method of embodiment 25, 26, 27, 28 or 29 wherein the responder patient has pre-diabetic hyperglycemia. In these embodiments, the pre-diabetic hyperglycemia may be ameliorated or progression of pre-diabetic hyperglycemia to diabetes may be slowed.

31. The method of embodiment 25, 26, 27, 28 or 29 wherein the responder patient has type 2 diabetes. Specific embodiments include responder patients having a BMI of at least 29.

32. The method of embodiment 25, 26, 27, 28 or 29 wherein the responder patient has type 1 diabetes. Specific embodiments include responder patients having a BMI of at least 29.

33. The method of embodiment 25, 26, 27, 28 or 29 wherein the responder patient has pre-diabetic hyperglycemia. Specific embodiments include responder patients having a BMI of at least 29.

34. The method of claim 25, 26, 27, 28 or 29 wherein the responder patient has a body mass index of at least 30.

35. A method to treat a patient(s) having diabetes, hyperglycemia, a macrovascular disease, a microvascular disease or a dyslipidemia condition, optionally hypercholesterolemia, comprising, (a) determining the body mass index (BMI) of the patient(s); (b) in a patient(s) of step (a) having a BMI of at least about 28, determining the level of fasting blood insulin and/or fasting blood C peptide; (c) selecting patient(s) with a fasting blood insulin level of at least about 4 μU/mL and/or a fasting blood C peptide level of at least about 2 ng/mL as responder patient(s); and (d) treating the responder patient(s) of step (c) with an effective amount of 17α-ethynylandrost-5-ene-3β,7β,17β-triol. In these embodiments, the compound used for treatment, e.g., 17α-ethynylandrost-5-ene-3β,7β,17β-triol, is preferably administered as an immediate release oral formulation, typically a tablet, capsule or gelcap. Such formulations will usually comprise two or more excipients and about 5 mg to about 50 mg of the compound. Preferred low unit doses of the compound, e.g., 17α-ethynylandrost-5-ene-313,7β,17β-triol, in these embodiments include about 2 mg (e.g., 2 mg±0.5 mg) and about 5 mg (e.g., 5 mg±1 mg), which can be used in adult and pediatric populations, e.g., about 2 mg administered once or twice per day or about 5 mg administered once or twice per day. Preferred standard unit doses of the compound, e.g., 17α-ethynylandrost-5-ene-3β,7β,17β-triol, in these embodiments include about 10 mg (e.g., 10 mg±2 mg), about 25 mg (e.g., 25 mg±5 mg) or about 50 mg (e.g., 50 mg±5 mg), which will be used primarily in adult populations, e.g., about 10 mg administered once or twice per day or about 25 mg administered once or twice per day. Other preferred standard unit doses of the compound, e.g., 17α-ethynylandrost-5-ene-3β,7β,17β-triol, in these embodiments include about 20 mg (e.g., 20 mg±2 mg) and about 45 mg (e.g., 45 mg±5 mg).

36. The method of embodiment 35 wherein the method further comprises determining the blood or serum level of MCP1 and selecting a patient(s) having a fasting blood insulin level of at least about 4 μU/mL and/or a fasting blood C peptide level of at least about 2 ng/mL as responder patient(s) for treatment in step (d). In these embodiments, the responder patient(s) can also have a MCP1 level of at least about 400 pg/mL, e.g., at least 500 pg/mL or at least 600 pg/mL. In these embodiments, the responder patient(s) can have a fasting blood insulin level of at least about 4.5 μU/mL and/or a fasting blood C peptide level of at least about 2.5 ng/mL.

37. The method of embodiment 35 or 36 wherein the responder patient(s) has a BMI of at least about 29. Responder patients include obese patients, i.e., patients having a BMI of at least about 30, which may be treated. In the responder patient(s), the patient(s) may have a waist circumference of about 40 inches (102 cm) or greater, which is associated with an increased risk of developing type 2 diabetes, hypertension and other cardiovascular disease such as stroke of myocardial infarction.

38. The method of embodiment 35, 36 or 37, wherein the responder patient(s) has type 2 diabetes and is treated with an effective amount of 17α-ethynylandrost-5-ene-3β,7β,17β-triol.

39. The method of embodiment 35, 36 or 37, wherein the responder patient(s) has hyperglycemia, optionally pre-diabetic hyperglycemia or, less preferably, hyperglycemia associated with a trauma or an infection, and is treated with an effective amount of 17α-ethynylandrost-5-ene-3β,7β,17β-triol.

40. The method of embodiment 35, 36 or 37, wherein the responder patient(s) has a microvascular disease and optionally hyperglycemia, wherein the microvascular disease optionally is retinopathy, neuropathy or nephropathy, and is treated with an effective amount of 17α-ethynylandrost-5-ene-3β,7β,17β-triol. The responder patient(s) in the embodiments described herein or in the claims as originally filed, e.g., in embodiment 35, 36, 37, 38, 39, 40, 41, 42, 43, 44, 45 or 46 will often have hyperglycemia (having a fasting serum glucose of at least about 110 mg/dL, usually at least about 140 mg/dL or as assessed by a clinician) or, less frequently, will be glucose intolerant glucose intolerant (having a fasting plasma glucose >120 and <126 mg/dL; 2-hr post prandial glucose 140-200 mg/dL after 75 g oral glucose). In these patients, the level of HbA1c will usually be elevated, e.g., at least 7.5%, and usually at least 8%. Elevated HbA1c reflects impaired glucose metabolism and contributes to the development and progression of microvascular disease including neuropathy. Elevated HbA1c also contributes to the development and progression of nephropathy.

41. The method of embodiment 35, 36 or 37, wherein the responder patient(s) has a macrovascular disease and optionally hyperglycemia, wherein the macrovascular disease optionally is atherosclerosis, arteriosclerosis, a stroke, hypertension or a myocardial infarction, and is treated with an effective amount of 17α-ethynylandrost-5-ene-3β,7β,17β-triol.

42. The method of embodiment 35, 36 or 37, wherein the responder patient(s) has a dyslipidemia condition and optionally hyperglycemia, wherein the dyslipidemia condition optionally is hypercholesterolemia or hypertriglyceridemia, and is treated with an effective amount of 17α-ethynylandrost-5-ene-3β,7β,17β-triol.

43. Use of an compound for the preparation of a medicament (a) for the treatment of diabetes, hyperglycemia, macrovascular disease, microvascular disease or a hyperlipidemia condition, optionally hypercholesterolemia in responder patient(s) or patient(s) having (i) a BMI of at least about 28 (e.g., at least 28), and (ii) a fasting blood insulin level of at least about 4 μU/mL and/or a fasting blood C peptide level of at least about 2 ng/mL, wherein medicament is 17α-ethynylandrost-5-ene-3β,7β,17β-triol. Responder patients preferably are (i) patients having a BMI of at least 28 and who are taking an insulin sensitizer such as metformin or (ii) patients having a BMI of at least 31 and who are not taking an insulin sensitizer such as metformin.

44. Use according to embodiment 43 wherein the responder patient(s) or patient(s) has (i) a blood serum MCP1 level of at least about 400 pg/mL, (ii) a BMI of at least about 29 or (iii) a blood serum MCP1 level of at least about 400 pg/mL and a BMI of at least about 29.

45. Use according to embodiment 43 or 44 wherein, the responder patient(s) or patient(s) has type 2 diabetes.

46. Use according to embodiment 43 or 44 wherein, the responder patient(s) or patient(s) has hyperglycemia, optionally pre-diabetic hyperglycemia or hyperglycemia associated with a trauma or an infection, and is treated with 17α-ethynylandrost-5-ene-3β,7β,17β-triol. In some embodiments, the responder patient(s) has pre-diabetic hyperglycemia, which can be treated according to the embodiments or original claims. In other embodiments, the responder patient(s) has hyperglycemia associated with a trauma or an infection, preferably an infection, which can be treated according to the embodiments, examples or original claims described herein. Such infections may be subsequent to a trauma, e.g., a wound such as a laceration or a surgical wound or a thermal burn.

47. Use according to embodiment 43 or 44 wherein, the responder patient(s) or patient(s) has a microvascular disease and optionally hyperglycemia, wherein the microvascular disease optionally is retinopathy, neuropathy or nephropathy. In some of these embodiments, the responder patient(s) has neuropathy, which can be treated according to the embodiments, examples or original claims and optionally which can be associated with or caused by hyperglycemia (or diabetes). In other embodiments, the responder patient(s) has nephropathy, which can be associated with or caused by hyperglycemia (or diabetes) and which can be treated according to the embodiments, examples or original claims described herein.

48. Use according to embodiment 43 or 44 wherein, the responder patient(s) or patient(s) has a macrovascular disease and optionally hyperglycemia, wherein the microvascular disease optionally is atherosclerosis or arteriosclerosis. In some of these embodiments, the responder patient(s) has atherosclerosis (or arteriosclerosis), which can be treated according to the embodiments, examples or original claims and optionally which can be associated with or caused by hyperglycemia (or diabetes) and/or (1) a BMI of 28, 29 or higher and/or (2) elevated MCP1 levels. In other embodiments, the responder patient(s) has experienced a stroke or myocardial infarction. Such stroke or myocardial infarction can be associated with or related to hyperglycemia (or diabetes) and treated according to the embodiments, examples or original claims described herein.

49. Use according to embodiment 43 or 44 wherein, the responder patient(s) or patient(s) has a macrovascular disease and optionally hyperglycemia, wherein the macrovascular disease is a stroke, hypertension or a myocardial infarction. In some of these embodiments, the responder patient(s) or patient(s) has atherosclerosis (or arteriosclerosis), which can be treated according to the embodiments, examples or original claims and optionally which can be associated with or caused by hyperglycemia (or diabetes) and/or (1) a BMI of 28, 29 or higher and/or (2) elevated MCP1 levels.

50. Use according to embodiment 43 or 44 wherein, the responder patient(s) or patient(s) has a dyslipidemia condition and optionally hyperglycemia, wherein the dyslipidemia condition, optionally is hyperlipidemia (optionally hypercholesterolemia or hypertriglyceridemia). In some of these embodiments, the responder patient(s) or patient(s) will have hypertriglyceridemia, which can be treated according to the embodiments, examples or original claims and optionally which can be associated with or caused by hyperglycemia (or diabetes) and/or (1) a BMI of 28, 29 or higher and/or (2) elevated MCP1 levels. In some of these embodiments, the responder patient(s) or patient(s) has hypercholesterolemia, e.g., elevated relative LDL cholesterol and/or decreased relative HDL cholesterol, which can be treated according to the embodiments, examples or original claims and optionally which can be associated with or caused by hyperglycemia (or diabetes) and/or (1) a BMI of 28, 29 or higher and/or (2) elevated MCP1 levels. In preferred embodiments, the responder patient(s) or patient(s) will have (a) hyperglycemia or (b) a BMI of 28, 29 or higher. In any of these embodiments, the responder patient(s) or patient(s) can have elevated MCP1 levels.

51. A method to treat hyperglycemia, diabetes, dyslipidemia, a macrovascular disease or a microvascular disease in a responder patient(s) or patient(s) in need thereof comprising administering an amount of 17α-ethynylandrost-5-ene-3β,7β,17β-triol sufficient to maintain a serum level of about 0.5 ng/mL to about 200 ng/mL for about 2 hours to at least about 4 hours. The patient(s) in these embodiments will usually have a fasting blood insulin level of at least about 4 μU/mL, usually have a fasting blood insulin level of at about 4 μU/mL to about 29 μU/mL, which is the normal range for insulin. Treatments for these conditions will typically be prolonged or chronic, e.g., for at least about 3 months or least about 6 months, because these diseases and conditions tend to be chronic. These diseases and conditions will tend to respond to treatment gradually. Serum levels of about 0.5 ng/mL to about 200 ng/mL are most conveniently attained for about 2 hours to at least about 4 hours using immediate release oral formulations that contain 17α-ethynylandrost-5-ene-3β,7β,17β-triol. Such dosages are preferably in the form of standard oral dosage forms, e.g., tablets, capsules or gelcaps, that can deliver about 4 mg/day to about 200 mg/day of the compound. Preferred unit doses for adults are 5 mg, 10 mg, 15 mg, 20 mg, 30 mg, 50 mg and 100 mg. Such unit doses can be administered once per day, e.g., for 20 mg, 30 mg, 50 mg or 100 mg doses, or twice per day for any of these. In terms of drug exposure per day (AUC₀₋₂₄), these dosages will provide 17α-ethynylandrost-5-ene-3β,7β,17β-triol drug exposure levels of about 15 ng·hr/mL to about 220 ng·hr/mL in adults.

52. The method of embodiment 51 wherein the amount of 17α-ethynylandrost-5-ene-3β,7β,17β-triol is sufficient to maintain a serum level of about 3 ng/mL to about 50 ng/mL for about 2 hours to at least about 4 hours. For drug exposure levels, a related embodiment is the method of embodiment 51 wherein the amount of 17α-ethynylandrost-5-ene-3β,7β,17β-triol is sufficient to provide 24 hour drug exposure levels per day (AUC₀₋₂₄) of about 50 ng·hr/mL to about 200 ng·hr/mL.

53. The method of embodiment 51 or 52 wherein about 4 mg to about 100 mg of 17α-ethynylandrost-5-ene-3β,7β,17β-triol in the form of a unit dose for oral administration, is administered each day as a single dose or as two doses. In the embodiments described herein and in the claims as originally filed, the oral dosages are usually in the form of immediate release oral formulations, e.g., tablets, capsules or gelcaps. Such formulations and dosages have been found to provide the serum levels that provide efficacy in patients that are treated. Preferred dosages for twice daily dosing may contain about 10 mg/dose or about 20 mg/dose for adults and about 2 mg/dose or about 5 mg/dose in pediatric populations. Dosages for once daily dosing may contain about 60 mg/dose and about 100 mg/dose for adults and about 10 mg/dose and about 20 mg/dose in pediatric populations.

54. The method of embodiment 51 or 52 wherein about 10 mg to about 60 mg of 17α-ethynylandrost-5-ene-3β,7β,17β-triol, optionally in the form of a unit dose for oral administration, is administered each day as a single dose or as two doses.

55. The method of embodiment 51 or 52 wherein the patient(s) has a BMI of at least about 28, has (i) a fasting blood insulin level of at least about 4 μU/mL and/or (ii) a fasting blood C peptide level of at least about 2 ng/mL, and optionally a fasting blood level of MCP1 of at least 400 pg/mL. In these embodiments, the patient(s) can have a BMI of at least about 29.

56. The method of embodiment 51 or 52 wherein, (a) the patient(s) has type 2 diabetes; or (b) the patient(s) has hyperglycemia, optionally pre-diabetic hyperglycemia or hyperglycemia associated with a trauma or an infection.

57. The method of embodiment 52 wherein, (a) the patient(s) has a microvascular disease and optionally hyperglycemia, wherein the microvascular disease optionally is retinopathy, neuropathy or nephropathy; (b) the patient(s) has a macrovascular disease and optionally hyperglycemia, wherein the macrovascular disease optionally is atherosclerosis, a stroke, hypertension or a myocardial infarction; or (c) the patient(s) has a dyslipidemia condition and optionally hyperglycemia, wherein the dyslipidemia condition optionally is hypercholesterolemia or hypertriglyceridemia.

58. A pharmaceutical formulation for oral administration comprising one or more excipients and 17α-ethynylandrost-5-ene-3β,7β,17β-triol in an amount sufficient to maintain a serum level in a patient of about 0.5 ng/mL to about 200 ng/mL for about 2 hours to at least about 4 hours. The serum levels are observed on days when the compound is administered. In these embodiments and others described herein, the formulation typically comprises two or more excipients, a lubricant such as sodium lauryl sulfate or magnesium stearate and other excipients such as microcrystalline cellulose or crospovidone and 17α-ethynylandrost-5-ene-33,7β,17β-triol. these formulations permit immediate release of the drug after oral ingestion.

59. A pharmaceutical formulation according to embodiment 58 wherein the amount of 17α-ethynylandrost-5-ene-3β,7β,17β-triol is sufficient to maintain a serum level of about 3 ng/mL to about 50 ng/mL for about 2 hours to at least about 4 hours.

60. A pharmaceutical formulation according to embodiment 58 or 59 wherein the pharmaceutical formulation is a unit dosage for oral administration and wherein each unit dosage contains about 2 mg, about 5 mg, about, 10 mg, about 25 mg, about 50 mg or about 100 mg of 17α-ethynylandrost-5-ene-3β,7β,17β-triol.

61. A pharmaceutical formulation according to embodiment 58 or 59 wherein the pharmaceutical formulation is a unit dosage for oral administration wherein each unit dosage contains about 15 mg, about 30 mg, about 45 mg or about 90 mg of 17α-ethynylandrost-5-ene-3β,7β,17β-triol.

62. A pharmaceutical formulation according to embodiment 58 or 59 wherein the pharmaceutical formulation is a unit dosage for oral administration wherein each unit dosage contains about 15 mg, about 30 mg, about 45 mg or about 90 mg of 17α-ethynylandrost-5-ene-3β,7β,17β-triol.

63. 17α-Ethynylandrost-5-ene-3β,7β,17β-triol for use in a therapy of diabetes, hyperglycemia, a macrovascular disease, a microvascular disease or a hyperlipidemia or dyslipidemia condition in a patient by administering an amount of 17α-ethynylandrost-5-ene-3β,7β,17β-triol to a patient sufficient to maintain a serum level in the patient of about 0.5 ng/mL to about 200 ng/mL for about 2 hours to at least about 4 hours per day.

64. Use according to embodiment 63 wherein the medicament is in the form of a unit dose for oral immediate release administration and contains about 2 mg to about 100 mg of 17α-ethynylandrost-5-ene-3β,7β,17β-triol. In these embodiments, the use or medicament preferably provides, about 10 mg to about 60 mg of 17α-ethynylandrost-5-ene-3β,7β,17β-triol per day.

65. Use according to embodiment 63 or 64 wherein the amount of 17α-ethynylandrost-5-ene-3β,7β,17β-triol is sufficient to maintain a serum level of about 3 ng/mL to about 50 ng/mL for about 2 hours to at least about 4 hours per day.

66. A pharmaceutical composition or dosage form comprising 17α-ethynylandrost-5-ene-3β,7β,17β-triol in an amount sufficient to maintain a serum level in a patient of about 0.5 ng/mL to about 200 ng/mL for about 2 hours to at least about 4 hours per day.

### Embodiment 67

A method to treat a patient(s) having diabetes, hyperglycemia, a macrovascular disease, a microvascular disease, nonalcoholic steatohepatitis, acute alcoholic hepatitis or a dyslipidemia condition, optionally hypercholesterolemia, comprising, (a) determining the body mass index (BMI) of the patient(s); (b) in a patient(s) of step (a) having a BMI of at least about 28, determining the level of fasting blood insulin and/or fasting blood C peptide; (c) selecting patient(s) with a fasting blood insulin level of at least about 4 μU/mL and/or a fasting blood C peptide level of at least about 2 ng/mL as patient(s) for treatment; and (d) treating the patient(s) of step (c) with an effective amount of 17α-ethynylandrost-5-ene-3β,7β,17β-triol. The patient(s) that have a fasting blood insulin level of at least about 4 μU/mL will usually have a fasting blood insulin level of at about 4 μU/mL to about 29 μU/mL, which is the normal range for insulin. Treatments for these conditions will typically be prolonged or chronic, e.g., for at least about 3 months or least about 6 months, because these diseases and conditions tend to be chronic. These diseases and conditions will tend to respond to treatment gradually.

68. The method of embodiment 67 wherein the method further comprises determining the blood or serum level of MCP1 and selecting a patient(s) having a MCP1 level of at least about 400 pg/mL and a fasting blood insulin level of at least about 4 μU/mL or, less preferably, a fasting blood C peptide level of at least about 2 ng/mL as patient(s) for treatment according to step (d).

69 The method of embodiment 67 or 68 wherein the patient(s) has a BMI of at least 29.

70. The method of embodiment 67 or 68 wherein, the patient(s) has type 2 diabetes.

71. The method of embodiment 67 or 68 wherein, the patient(s) has hyperglycemia, optionally pre-diabetic hyperglycemia or hyperglycemia associated with a trauma or an infection. In related embodiments, the patient(s) will have pre-diabetic hyperglycemia. In other related embodiments, the patient(s) will have hyperglycemia and type 2 diabetes.

72. The method of embodiment 67 or 68 wherein, the patient(s) has a microvascular disease and optionally hyperglycemia, wherein the microvascular disease optionally is retinopathy, neuropathy or nephropathy. In related embodiments, the patient(s) will have hyperglycemia. In other related embodiments, the patient(s) will have type 2 diabetes.

73. The method of embodiment 67 or 68 wherein, the patient(s) has a macrovascular disease and optionally hyperglycemia, wherein the macrovascular disease optionally is atherosclerosis, arteriosclerosis, a stroke, hypertension or a myocardial infarction. In related embodiments, the patient(s) will have hyperglycemia. In other related embodiments, the patient(s) will have hyperglycemia and type 2 diabetes.

74. The method of embodiment 67 or 68 wherein, the patient(s) has a dyslipidemia condition and optionally hyperglycemia, wherein the dyslipidemia condition optionally is hypercholesterolemia or hypertriglyceridemia, and is treated with an effective amount of 17α-ethynylandrost-5-ene-3β,7β,17β-triol. In related embodiments, the patient(s) will have hyperglycemia. In other related embodiments, the patient(s) will have hyperglycemia and type 2 diabetes.

75. The method of embodiment 67 or 68 wherein, the patient(s) has a nonalcoholic steatohepatitis or alcoholic hepatitis and optionally hyperglycemia, and is treated with an effective amount of 17α-ethynylandrost-5-ene-3β,7β,17β-triol. In related embodiments, the patient(s) will have hyperglycemia. In other related embodiments, the patient(s) has hyperglycemia or type 2 diabetes. In related embodiments, the patient(s) has nonalcoholic steatohepatitis.

76. The method of embodiment 67 or 68 wherein, about 10 mg/day to about 200 mg/day of 17α-ethynylandrost-5-ene-3β,7β,17β-triol is administered to the patient(s).

### Embodiment 77

A pharmaceutical formulation for oral administration comprising one or more excipients and 17α-ethynylandrost-5-ene-3β,7β,17β-triol in an amount sufficient to maintain a serum level in a patient of about 0.5 ng/mL to about 200 ng/mL for about 2 hours to at least about 4 hours, preferably about 3 ng/mL to about 50 ng/mL for about 2 hours to at least about 4 hours.

78. A pharmaceutical formulation according to embodiment 77 wherein the pharmaceutical formulation is a unit dosage for oral administration wherein each unit dosage contains about 2 mg, about 5 mg, about, 10 mg, about 25 mg, about 50 mg or about 100 mg of 17α-ethynylandrost-5-ene-3β,7β,17β-triol.

79. A pharmaceutical formulation according to claim embodiment 77 wherein the pharmaceutical formulation is a unit dosage for oral administration wherein each unit dosage contains about 15 mg, about 30 mg, about 45 mg, about 60 mg or about 90 mg of 17α-ethynylandrost-5-ene-3β,7β,17β-triol.

80. A pharmaceutical formulation according to claim embodiment 77, 78 or 79 wherein the pharmaceutical formulation is an immediate release formulation. Exemplary immediate release formulations are usually used (preferred) in the claims as originally filed or in the methods described elsewhere herein, e.g., in embodiments 1, 2, 17, 18, 25, 26, 35, 36, 40, 43, 51, 52 and other embodiments that refer to these embodiments. Exemplary immediate release formulations are described in some of the following examples, e.g., examples 3 and 9.

81. A treatment method for a patient(s), comprising, (a) determining the body mass index (BMI) of the patient(s); (b) in a patient(s) of step (a) having a BMI of at least about 31 (preferably at least 30.5), determining the level of fasting blood insulin and/or a fasting blood C peptide level; (c) selecting patient(s) with a fasting blood insulin level of at least about 4 μU/mL as patient(s) for treatment and/or a fasting blood C peptide level of at least about 2 ng/mL; and (d) treating the patient(s) of step (c) with an effective amount of 17α-ethynylandrost-5-ene-3β,7β,17β-triol, wherein the patient(s) have one or more of hyperglycemia, diabetes, a macrovascular disease, a microvascular disease, nonalcoholic fatty liver disease, nonalcoholic steatohepatitis, acute alcoholic hepatitis or a dyslipidemia condition and wherein the patient(s) has not been treated with metformin, buformin or phenformin and optionally wherein the patient(s) has not been treated with another antidiabetic agent or another insulin sensitizer. Metformin, buformin, phenformin, pioglitazone and rosiglitazone are insulin sensitizers that are included in this embodiment. Buformin and phenformin are rarely used due to toxicity. Metformin is the most preferred insulin sensitizer that has not been administered to the patient(s) in this embodiment. Less preferred insulin sensitizers that have not been administered are pioglitazone and rosiglitazone.

82. The method of embodiment 81 wherein the patient(s) has a BMI of at least about 32, preferably at least 31.7.

83. The method of embodiment 81 or 82 wherein the method further comprises determining the blood or serum level of MCP1 and selecting a patient(s) having a MCP1 level of at least about 400 pg/mL and a fasting blood insulin level of at least about 4 μU/mL as patient(s) for treatment according to step (d).

84. The method of embodiment 81 or 82 wherein, the patient(s) has type 2 diabetes.

85. The method of embodiment 81 or 82 wherein, the patient(s) has type 1 diabetes.

86. The method of embodiment 81 or 82 wherein, the patient(s) has pre-diabetic hyperglycemia.

87. The method of embodiment 81 or 82 wherein, the patient(s) has a microvascular disease and optionally hyperglycemia, wherein the microvascular disease optionally is retinopathy, neuropathy or nephropathy.

88. The method of embodiment 81 or 82 wherein, the patient(s) has a macrovascular disease and optionally hyperglycemia, wherein the macrovascular disease optionally is atherosclerosis, arteriosclerosis, a stroke, hypertension or a myocardial infarction.

89. The method of embodiment 81 or 82 wherein, the patient(s) has a dyslipidemia condition and optionally hyperglycemia, wherein the dyslipidemia condition is hypercholesterolemia or hypertriglyceridemia.

90. The method of embodiment 81 or 82 wherein, the patient(s) has hepatitis, optionally nonalcoholic steatohepatitis and optionally hyperglycemia.

91. The method of embodiment 81 or 82 wherein, the patient(s) has nonalcoholic fatty liver disease and optionally hyperglycemia.

92. The method of embodiment 81 or 82 wherein, the patient(s) has acute alcoholic hepatitis and optionally hyperglycemia.

93. The compound 17α-ethynylandrost-5-ene-3β,7β,17β-triol for use in the treatment of hyperglycemia, diabetes, a macrovascular disease, a microvascular disease, nonalcoholic fatty liver disease, nonalcoholic steatohepatitis, acute alcoholic hepatitis or a dyslipidemia condition in a patient(s) having (i) a BMI of at least 31, (ii) a fasting blood insulin level of at least about 4 μU/mL and/or a fasting blood C peptide level of at least about 2 ng/mL and (iii) no prior treatment with metformin, buformin or phenformin and optionally another antidiabetic agent.

94. Use according to embodiment 93 wherein the patient(s) has a BMI of at least 32.

95. Use according to embodiment 93 wherein the patient(s) has a BMI of at least 33.

96. Use according to embodiment 93, 94 or 95 wherein, the compound is for the treatment of type 2 diabetes.

97. Use according to embodiment 93, 94 or 95 wherein, the compound is for the treatment of type 1 diabetes.

98. Use according to embodiment 93, 94 or 95 wherein, the compound is for the treatment of pre-diabetic hyperglycemia.

99. Use according to embodiment 93, 94 or 95 wherein, the compound is for the treatment of a microvascular disease, wherein the microvascular disease optionally is retinopathy, neuropathy or nephropathy.

100. Use according to embodiment 93, 94 or 95 wherein, the compound is for the treatment of a macrovascular disease, wherein the macrovascular disease optionally is atherosclerosis, atherosclerosis, a stroke, hypertension or a myocardial infarction.

101. Use according to embodiment 93, 94 or 95 wherein, the compound is for the treatment of a dyslipidemia condition, wherein the dyslipidemia condition is hypercholesterolemia or hypertriglyceridemia.

102. Use according to embodiment 93, 94 or 95 wherein, the compound is for the treatment of hepatitis, optionally nonalcoholic steatohepatitis.

103. Use according to embodiment 93, 94 or 95 wherein, the compound is for the treatment of acute alcoholic hepatitis.

104. Use according to embodiment 93, 94 or 95 wherein, the compound is for the treatment of nonalcoholic fatty liver disease.

105. Use according to embodiment 93, 94, 95, 96, 97, 98, 99, 100, 101, 102, 103 or 104 wherein the patient(s) has (i) a blood serum MCP1 level of at least 400 pg/mL and a fasting blood insulin level of at least 4 μU/mL, (ii) a BMI of at least 32.3 or (iii) a blood serum MCP1 level of at least 400 pg/mL, a fasting blood insulin level of at least about 4 μU/mL and a BMI of at least 33.

106. A method to treat a patient(s) in need thereof comprising administering an amount of 17α-ethynylandrost-5-ene-3β,7β,17β-triol sufficient to maintain a serum level of about 0.5 ng/mL to about 200 ng/mL for about 2 hours to at least about 4 hours per day, wherein the patient(s) has hyperglycemia, diabetes, dyslipidemia, a macrovascular disease, a microvascular disease, nonalcoholic fatty liver disease, hepatitis, optionally selected from the group consisting of nonalcoholic steatohepatitis, viral hepatitis and acute alcoholic hepatitis and wherein the patient(s) has a BMI of at least 31 and has not previously been treated with metformin and optionally another antidiabetic agent. In this and other embodiments described herein, viral hepatitis means hepatitis associated with an infection caused by a hepatitis virus, e.g., hepatitis C virus, hepatitis B virus or hepatitis A virus.

107. The method of embodiment 106 wherein the amount of 17α-ethynylandrost-5-ene-3β,7β,17β-triol is sufficient to maintain a serum level of about 3 ng/mL to about 50 ng/mL for at least 4 hours.

108. The method of embodiment 106 or 107 wherein about 4 mg to about 250 mg of 17α-ethynylandrost-5-ene-3β,7β,17β-triol in the form of a dose for oral administration that is administered each day as a single dose or twice as two doses each containing about 2 mg per dose to about 100 mg per dose. Such doses are preferably unit doses, e.g., tablets, capsules or gelcaps. Exemplary preferred oral dosages are 100 mg and 150 mg. More preferred oral dosages are 200 mg and 250 mg. Other useful oral dosages are 400 mg and 500 mg.

109. The method of embodiment 106 or 107 wherein about 10 mg to about 150 mg of 17α-ethynylandrost-5-ene-3β,7β,17β-triol is administered in the form of a unit dose for oral administration, optionally wherein the 17α-ethynylandrost-5-ene-3β,7β,17β-triol is administered each day as a single dose or twice as two doses each containing about 5 mg per dose to about 75 mg per dose. Such doses are preferably unit doses, e.g., tablets, capsules or gelcaps.

110. The method of embodiment 106, 107, 108 or 109 wherein patient(s) has a BMI of at least 32.

111. The method of embodiment 106, 107, 108 or 109 wherein patient(s) has a BMI of at least 33.

112. The method of embodiment 106, 107, 108, 109, 110 or 111 wherein, (a) the patient(s) has type 2 diabetes; (b) the patient(s) has type 1 diabetes; or (c) the patient(s) has pre-diabetic hyperglycemia.

113. The method of embodiment 106, 107, 108, 109, 110 or 111 wherein, (a) the patient(s) has a microvascular disease and optionally hyperglycemia, wherein the microvascular disease optionally is retinopathy, neuropathy or nephropathy; or (b) the patient(s) has a macrovascular disease and optionally hyperglycemia, wherein the macrovascular disease optionally is atherosclerosis, arteriosclerosis, hypertension, a thromboembolism, a stroke or a myocardial infarction.

114. The method of embodiment 106, 107, 108, 109, 110 or 111 wherein, (a) the patient(s) has a dyslipidemia condition and optionally hyperglycemia, wherein the dyslipidemia condition optionally is hypercholesterolemia or hypertriglyceridemia; (b) the patient(s) has nonalcoholic steatohepatitis and optionally hyperglycemia; (c) the patient(s) has hepatitis, optionally acute alcoholic hepatitis and optionally hyperglycemia; or (d) the patient(s) has nonalcoholic fatty liver disease and optionally hyperglycemia.

115. The method of embodiment 106, 107, 108, 109, 110, 111, 112, 113 or 114 wherein, the patient(s) has a BMI of at least 32, has (i) a fasting blood insulin level of at least about 4 μU/mL and/or (ii) a fasting blood C peptide level of at least about 2 ng/mL, and optionally a blood level of MCP1 of at least about 400 pg/mL.

116. The method or use of embodiment 81, 82, 83, 87, 88, 89, 90, 91, 102, 113, 114 or 115, wherein the patient(s) has hyperglycemia.

117. A method to treat hyperglycemia, diabetes, dyslipidemia, a macrovascular disease, a microvascular disease, hepatitis, optionally nonalcoholic steatohepatitis, acute alcoholic hepatitis or viral in a patient(s) in need thereof comprising administering an amount of 17α-ethynylandrost-5-ene-3β,7β,17β-triol sufficient to maintain a serum level of about 0.5 ng/mL to about 200 ng/mL for about 2 hours per day to at least about 4 hours per day, wherein (a) the patient(s) has a body mass index (BMI) of at least 28 or a BMI of at least 29 and the patient(s) is also treated with metformin or another insulin sensitizer or (b) the patient(s) has a BMI of at least 30, at least 31 or at least 32 and the patient(s) is not treated with metformin or another insulin sensitizer. This embodiment includes patient(s) having a BMI of at least 30, wherein the patient(s) is not treated with metformin or another insulin sensitizer. This embodiment preferably includes patient(s) having a BMI of at least 31 or at least 32 wherein the patient(s) is not treated with metformin or another insulin sensitizer.

118. The method of embodiment 117 wherein (a) the amount of 17α-ethynylandrost-5-ene-3β,7β,17β-triol is sufficient to maintain a serum level of about 3 ng/mL to about 50 ng/mL for about 2 hours per day to at least about 4 hours per day; (b) about 4 mg to about 200 mg of 17α-ethynylandrost-5-ene-3β,7β,17β-triol in the form of a dose for oral administration that is administered each day as a single dose or twice as two doses containing about 2 mg per dose to about 100 mg per dose; or (c) about 10 mg to about 100 mg of 17α-ethynylandrost-5-ene-3β,7β,17β-triol is administered in the form of a unit dose for oral administration, optionally wherein the 17α-ethynylandrost-5-ene-3β,7β,17β-triol is administered each day as a single dose or twice as two doses containing about 5 mg per dose to about 50 mg per dose.

119. A method to treat nonalcoholic fatty liver disease or hepatitis in a patient in need thereof comprising administering an effective amount of 17α-ethynylandrost-5-ene-3β,7β,17β-triol.

120. The method of embodiment 119 wherein the hepatitis is nonalcoholic steatohepatitis, viral hepatitis or acute alcoholic hepatitis.

121. The method of embodiment 120 or 121 wherein the 17α-ethynylandrost-5-ene-3β,7β,17β-triol is administered as described in embodiment 118, or wherein 200 mg/day to 750 mg/day of 17α-ethynylandrost-5-ene-3β,7β,17β-triol is administered.

122. A method to treat a patient(s) having type 2 diabetes or pre-diabetic hyperglycemia, comprising, (a) determining the body mass index (BMI) of the patient(s) and selecting patient(s) having a BMI of >28, e.g., >28.5 or >29; (b) determining the fasting blood glucose level or the post prandial glucose level in the patients of step (a); (c) selecting patients with a fasting blood glucose level of at least 110 mg/dL or a post prandial glucose level of at least about 140 mg/dL as patient(s) for treatment; and (d) treating the patient(s) of step (c) with an effective amount of 17α-ethynylandrost-5-ene-3β,7β,17β-triol, wherein the patient(s) are also treated with an anti type 2 diabetic or anti-hyperglycemic effective amount of metformin, optionally 500 mg/day to 2000 mg/day.

123. The method of embodiment 122 wherein the patient(s) has a BMI of at least 30 or 30.5.

124. The method of embodiment 122 wherein the patient(s) has a BMI of at least 31.

125. The method of embodiment 122, 123 or 124 wherein, the patients of step (c) have a fasting insulin level of at least 4 μU/mL.

126. The method of embodiment 122, 123, 124 or 125 wherein, the patients of step (c) have (i) a fasting blood glucose level of at least 120 mg/dL or a post prandial glucose level of at least about 150 mg/dL or (ii) a fasting blood glucose level of at least 126 mg/dL or a post prandial glucose level of at least about 200 mg/dL.

### Examples

The following examples further illustrate the invention and they are not intended to limit it in any way. Variations of these examples that are included in the invention may include, e.g., modification of a step(s) of any of the methods described herein.

### Example 1

### Clinical Trial Cohorts

Groups of pre-diabetic hyperglycemic patients and patients having type 2 diabetes were treated for 28 days with the experimental drug 17α-ethynylandrost-5-ene-3β,7β,17β-triol (oral capsules) or with placebo. Dosages for the pre diabetic patients were 0 mg per day (placebo control; n=10), 5 mg once per day (n=9; cohort 1), 5 mg twice per day (n=8; cohort 2) or 10 mg twice per day (n=7; cohort 3). Placebos were obtained with each treatment cohort based on protocol randomization of 2 treated:1 placebo. The pre-diabetic patients were obese (body mass index ≧29 kg/m²) and glucose intolerant (fasting plasma glucose <126 mg/dL; 2-hr post prandial glucose 140-200 mg/dL after 75 g oral glucose). The diabetic patients were dosed with 10 mg/day for 28 days (n=6; cohort 6). The glucose clamp protocol was performed on groups of patients dosed with 10 mg/day or 20 mg/day of drug at baseline, one day before drug treatment started (day −1), and one day after the last day of dosing with the drug (day 29). Patients with an M value of <5 mg glucose/kg/min (M<5) were considered insulin resistant and patients with an M value of >5 mg glucose/kg/min (M>5) were considered insulin sensitive or essentially normal for whole body glucose metabolism.

Levels of cytokine biomarkers from patients with M values measured by glucose clamp such as MCP1, TNFα, IL-6 and IL-1β were measured by ELISA as the amount of cytokine secreted from LPS stimulated peripheral blood mononuclear cells from the patients at baseline (day −1) and at 1 day after the last day of drug dosing (day 29). Levels of RBP4 and CRP protein were measured from patient serum.

### Example 2

### Analysis of Biomarkers and Response to Treatment

The analysis of responders used standard analysis. Due to the cohort sizes and the ongoing basis of this clinical protocol, data for surrogates that corresponded to significance at p≦0.10 (trend) were considered positive evidence of surrogacy of the biomarker for the M<5 insulin resistant condition.

A summary of results included the following observations. CRP significantly decreased in the drug treated insulin resistant subjects (M<5) compared to placebo-treated subjects (p=0.018 exact Mann-Whitney). The decrease in CRP in M<5 patients at baseline was significant compared to the M>5 patients (p=0.094 exact Mann-Whitney).

Cytokines TNFα (p=0.0032 exact Mann-Whitney), IL-6 (p=0.0265 exact Mann-Whitney), MCP1 (p=0.054 exact Mann-Whitney), and IL-113 (p=0.0401 exact Mann-Whitney) were elevated at baseline in M<5 patients compared to the M>5 patients. There was a greater decrease at day 29 in MCP1 (p=0.093) and TNFα (p=0.075) in the insulin resistant patients (M<5) that were treated with drug than in insulin sensitive patients (M>5) that were treated with drug.

Initial resistin levels were more elevated in M<5 patients than M>5 cohorts 2 & 3 and in M<5 than M>5 Cohort 6. Resistin rose in placebo control patients and increased less in drug treated subjects, and it increased much less in M<5 drug treated subjects. Leptin decreased in placebo patients, but increased across all drug treated cohorts, and significantly increased in cohort 6 (p=0.002). Biomarkers that were not affected in the M<5 drug treated patients included the adipokine adiponectin.

Comparison of the effect of 17α-ethynylandrost-5-ene-3β,7β,17β-triol treatment of cohort 2, 3 and 6 M<5 patients vs. M>5 patients in cohort 2, 3 and 6 showed a highly significant decrease in IL-1β, IL-6, MCP1 and TNFα (p<0.03; LPS stimulated PBMC), which indicated that these biomarkers responded to a greater extent in the M<5 patients compared to the M>5 patients.

### Example 3

### Oral 17α-ethynylandrost-5-ene-3β,7β,17β-triol Oral Dosages

Oral dosage capsules containing 5 mg of 17α-ethynylandrost-5-ene-3β,7β,17β-triol were prepared. The formulation contained 5 mg micronized 17α-ethynylandrost-5-ene-3β,7β,17β-triol, 25 mg sodium lauryl sulfate, 74 mg microcrystalline cellulose (Avicel PH 102), 45 mg crospovidone (polyplasdone XL 10) and 1 mg magnesium stearate in hard gelatin capsules size #2 capsules with a fill weight of 150 mg.

Oral dosage capsules containing 1 mg of 17α-ethynylandrost-5-ene-3β, 7β,17β-triol were prepared. The formulation contained 1 mg micronized 17α-ethynylandrost-5-ene-3β,7β,17β-triol, 5 mg sodium lauryl sulfate, 98 mg microcrystalline cellulose (Avicel PH 102), 45 mg crospovidone (polyplasdone XL 10), 1 mg magnesium stearate in hard gelatin capsule size #2 capsules with a fill weight of 150 mg.

Oral dosage capsules containing 250 μg of 17α-ethynylandrost-5-ene-3β, 7β,17β-triol are prepared. The formulation contains 250 μg of micronized 17α-ethynylandrost-5-ene-3β,7β,17β-triol, 5 mg sodium lauryl sulfate, 99 mg microcrystalline cellulose (Avicel PH 102), 45 mg crospovidone (polyplasdone XL 10), 1 mg magnesium stearate in hard gelatin capsule size #2 capsules with a fill weight of 150 mg.

### Example 4

Patients having type 2 diabetes were evaluated for their baseline BMI and levels of MCP1, insulin and C peptide. The effect of treatment with 17α-ethynylandrost-5-ene-3β,7β,17β-triol (5 mg administered orally twice per day for 84 days) on total cholesterol, LDL cholesterol, hemoglobin A1c (HbA1c) plus metformin (500-2250 mg/day, which was administered in one or two doses per day for 84 days), weight, and other biological responses is shown in the tables below. The effect of treatment in all treated patients was compared with the effect of the same treatment in the responder population, characterized as having a BMI>27.99 (overweight or obese), insulin resistant (C-peptide >1.99 ng/mL or essentially normal insulin >3.99 μU/mL to about 29 μU/mL with hyperglycemia), and having an elevated serum MCP1>399.99 pg/mL, indicating a state of systemic inflammation. The responder patients were a subset of all patients were treated.

For insulin measurements, 1 international unit (IU or usually U) is the biological equivalent of about 45.5 pg pure crystalline insulin. As used herein, normal fasting insulin levels are considered to be about 4-29 μU/mL. There is some variation in the range of normal fasting insulin levels. One source provides the range of about 5-25 μU/mL, which is about 36-179 pmol/mL (laboratory reference value from Stedman's Concise Medical Dictionary, 3^{rd} Ed., 1997, page 992, Williams & Wilkins). Sufficiently elevated insulin levels, e.g., about 100 μU/mL or greater, are pathological, e.g., usually associated with coma or death. Normal insulin levels defined by other sources may vary somewhat, e.g., 26 μU/mL may be considered the upper limit of normal and 4 μU/mL or 6 μU/mL may be considered the lower limit of normal.

All patients—HbA1c Change from baseline (% Hb)

| | S/B* | 29 | 57 | 84** |
|---|---|---|---|---|
| treated | 0 | 0 | 0.1 | 0.15 |
| n | 35 | 35 | 33 | 32 |
| placebo | 0 | −0.2 | −0.25 | −0.05 |
| n | 41 | 39 | 41 | 34 |

*status at baseline, before dosing
**status at day 84, end of dosing

Responder patients—HbA1c Change from baseline (% Hb)

| | S/B | 29 | 57 | 84 |
|---|---|---|---|---|
| treated | 0 | −0.15 | −0.1 | −0.425 |
| n | 13 | 13 | 12 | 12 |
| placebo | 0 | −0.2 | −0.05 | 0.15 |
| n | 15 | 14 | 15 | 11 |

All patients—Fructosamine Change from baseline (mmol/mL)

| | S/B | 29 | 57 | 84 |
|---|---|---|---|---|
| treated | 0 | −2 | −1 | −12.75 |
| n | 35 | 35 | 35 | 24 |
| placebo | 0 | −10 | −14.5 | −12 |
| n | 40 | 38 | 39 | 24 |

Responder patients—Fructosamine Change from baseline (mmol/mL)

| | S/B | 29 | 57 | 84 |
|---|---|---|---|---|
| treated | 0 | 9 | −24 | −34 |
| n | 13 | 13 | 13 | 7 |
| placebo | 0 | −5.75 | −5.25 | −13 |
| n | 15 | 14 | 14 | 9 |

All patients—Fasting Glucose Change from baseline (mg/dL)

| | S/B | 29 | 57 | 84 |
|---|---|---|---|---|
| treated | 0 | −7.5 | 0 | −4.25 |
| n | 35 | 35 | 35 | 28 |
| placebo | 0 | −5.5 | 5.75 | −6.5 |
| n | 40 | 39 | 40 | 25 |

Responder patients—Fasting Glucose Change from baseline (mg/dL)

| | S/B | 29 | 57 | 84 |
|---|---|---|---|---|
| treated | 0 | −17.5 | −7 | −13 |
| n | 13 | 13 | 13 | 9 |
| placebo | 0 | −6.25 | 7 | 1.5 |
| n | 15 | 14 | 15 | 9 |

All patients—Fasting C-peptide Change from baseline (ng/mL)

| | S/B | 29 | 57 | 84 |
|---|---|---|---|---|
| treated | 0 | −0.2 | −0.2 | −0.05 |
| n | 35 | 35 | 35 | 24 |
| placebo | 0 | 0.225 | 0.125 | −0.075 |
| n | 40 | 38 | 38 | 24 |

Responder patients—Fasting C-peptide Change from baseline (ng/mL)

| | S/B | 29 | 57 | 84 |
|---|---|---|---|---|
| treated | 0 | −0.2 | 0 | 0.25 |
| n | 13 | 13 | 13 | 7 |
| placebo | 0 | −0.05 | 0.1 | 0.5 |
| n | 15 | 14 | 13 | 9 |

All patients—Fasting Insulin Change from baseline (mU/mL)

| | S/B | 29 | 57 | 84 |
|---|---|---|---|---|
| treated | 0 | −0.6 | −0.25 | −0.05 |
| n | 35 | 35 | 34 | 24 |
| placebo | 0 | 0.35 | 0.65 | −0.275 |
| n | 40 | 38 | 38 | 24 |

Responder patients—Fasting Insulin Change from baseline (mU/mL)

| | S/B | 29 | 57 | 84 |
|---|---|---|---|---|
| treated | 0 | 0 | 0.3 | −0.6 |
| n | 13 | 13 | 13 | 7 |
| placebo | 0 | 0.225 | 3.4 | 0.9 |
| n | 15 | 14 | 13 | 9 |

All patients —HOMA2% β cell function change from baseline (% b; calculated using C-peptide)

| | S/B | 29 | 57 | 84 |
|---|---|---|---|---|
| treated | 0 | 0.8 | −2.15 | 5.6 |
| n | 35 | 24 | 24 | 24 |
| placebo | 0 | 8.8 | −0.9 | 2.65 |
| n | 40 | 23 | 24 | 24 |

Responder patients—HOMA2% β cell function change from baseline (% b; calculated using C-peptide)

| | S/B | 29 | 57 | 84 |
|---|---|---|---|---|
| treated | 0 | 2.1 | 5.3 | 16.3 |
| n | 13 | 7 | 7 | 7 |
| placebo | 0 | 8.95 | −6 | 6.6 |
| n | 15 | 8 | 9 | 9 |

All patients—HOMA2% insulin sensitivity change from baseline (% S; calculated using C-peptide)

| | S/B | 29 | 57 | 84 |
|---|---|---|---|---|
| treated | 0 | 4.2 | 1.75 | −1.45 |
| n | 35 | 24 | 24 | 24 |
| placebo | 0 | −3 | −2.7 | 1.2 |
| n | 40 | 24 | 25 | 24 |

Responder patients—HOMA2% insulin sensitivity change from baseline (% S; calculated using C-peptide)

| | S/B | 29 | 57 | 84 |
|---|---|---|---|---|
| treated | 0 | 3.9 | 1 | −4.4 |
| n | 13 | 7 | 7 | 7 |
| placebo | 0 | 2.6 | −2.7 | −7.2 |
| n | 15 | 8 | 9 | 9 |

All patients—HOMA2 Insulin resistance change from baseline (IR; calculated using C-peptide)

| | S/B | 29 | 57 | 84 |
|---|---|---|---|---|
| treated | 0 | −0.14533 | −0.0518 | 0.023089 |
| n | 35 | 24 | 24 | 24 |
| placebo | 0 | 0.087096 | 0.108539 | −0.05977 |
| n | 40 | 23 | 24 | 24 |

Responder patients—HOMA2 Insulin resistance change from baseline (IR; calculated using C-peptide)

| | S/B | 29 | 57 | 84 |
|---|---|---|---|---|
| treated | 0 | −0.30606 | −0.04016 | 0.226519 |
| n | 13 | 7 | 7 | 7 |
| Placebo | 0 | −0.23216 | 0.704458 | 0.899137 |
| n | 15 | 8 | 9 | 9 |

All patients—Weight change from baseline (kg)

| | S/B | 29 | 57 | 84 |
|---|---|---|---|---|
| treated | 0 | −0.41364 | −0.45455 | −0.03864 |
| n | 35 | 32 | 30 | 22 |
| placebo | 0 | −0.20909 | −0.45455 | −0.56818 |
| n | 40 | 37 | 36 | 20 |

Responder patients—Weight change from baseline (kg)

| | S/B | 29 | 57 | 84 |
|---|---|---|---|---|
| treated | 0 | −0.37273 | −0.925 | −1.32955 |
| n | 13 | 13 | 12 | 6 |
| placebo | 0 | 0 | −0.28182 | 0 |
| n | 15 | 15 | 12 | 7 |

All patients—Total Cholesterol change from baseline (mg/dL)

| | S/B | 29 | 57 | 84 |
|---|---|---|---|---|
| treated | 0 | −0.5 | −5.5 | 3 |
| n | 35 | 35 | 35 | 27 |
| placebo | 0 | 3.5 | −3.5 | −2.5 |
| n | 39 | 39 | 35 | 21 |

Responder patients—Total Cholesterol change from baseline (mg/dL)

| | S/B | 29 | 57 | 84 |
|---|---|---|---|---|
| treated | 0 | −0.5 | −18.5 | −14.5 |
| n | 13 | 13 | 13 | 7 |
| placebo | 0 | 8 | −5.75 | 1 |
| n | 15 | 15 | 12 | 7 |

All patients—LDL Cholesterol change from baseline (mg/dL)

| 1 | S/B | 29 | 57 | 84 |
|---|---|---|---|---|
| treated | 0 | 0.5 | −2 | 4.5 |
| n | 35 | 35 | 35 | 27 |
| placebo | 0 | 3 | −3.5 | −0.5 |
| n | 39 | 39 | 35 | 19 |

Responder patients—LDL Cholesterol change from baseline (mg/dL)

| | S/B | 29 | 57 | 84 |
|---|---|---|---|---|
| treated | 0 | −8.5 | −11.5 | −7.5 |
| n | 13 | 13 | 13 | 7 |
| placebo | 0 | 5.5 | −2.25 | 4.5 |
| n | 15 | 15 | 12 | 5 |

The results shown above indicated that compared to all treated patients, the responder patient subset showed an improved response to treatment with 17α-ethynylandrost-5-ene-3β,7β,17β-triol plus metformin. The responder patients showed a greater decrease in HbA1c and fructosamine from baseline by the end of dosing at 84 days compared to the entire treated patient population. Those decreases indicate better control of glucose levels in the treated patients. Similarly, the responder patients showed a greater decrease in fasting glucose, fasting insulin, total cholesterol and LDL cholesterol indicating better regulation of glucose metabolism and improved cholesterol control. The greater increase in weight loss in the responder patients compared to all patients reflects improved glucose utilization by peripheral tissues (muscle, brain) and less diversion into synthesis and storage of fat in adipose tissue. Insulin sensitivity was similar in both the total treated population and the responder subset but insulin resistance was decreased in the responder subset. Increased % β cell (pancreatic beta cell) function in the responder patients compared to the total population showed an improved efficiency of the pancreatic response to glucose and thus better glucose regulation and use. Collectively, the biological response data in the responder patients indicated that this patient phenotype was qualitatively more responsive than the total population to treatment with 17α-ethynylandrost-5-ene-3β,7β,17β-triol in patients with glucose levels that were not adequately controlled by metformin.

### Example 5

Data obtained from pre-diabetic hyperglycemic patients described in example 1 indicated that treatment with 17α-ethynylandrost-5-ene-3β,7β,17β-triol was most effective in patients having an M<5 value as obtained from glucose clamp analysis.

Interim analysis. Placebo patients from that protocol had a change in M values from slightly less than 0 to about −2.2, indicating a generally decreased ability of the patients to utilize glucose. By contrast, patients treated with 10 mg/day (5 mg administered orally twice per day) showed a significantly increased M value (p=0.0286, Mann-Whitney) and pooled patients treated with 10 mg/day or 20 mg/day (10 mg administered orally twice per day) showed a significantly increased M value (p=0.0095, Mann-Whitney). Two patients treated with 20 mg/day, a sample size too small from which to obtain statistical data, had an increased M value. By contrast, in patients having essentially normal glucose utilization as reflected by an M>5, there was no statistically significant difference in a change in M value between placebo treated patients and patients treated with 10 mg/day, 20 mg/day or pooled treated patients. In the overall protocol with no consideration for M value subgroups, the pooled patients had a significantly increased M value (p=0.0278, Mann-Whitney), with trends to significance in the mg/day group (p=0.0513, Mann-Whitney) and in the 20 mg/day group (p=0.0823, Mann-Whitney).

Second interim analysis. Placebo patients from that protocol had a change in M values from slightly less than 0 to about −2.9 with a median of −1.22 from baseline. Patients treated with 10 mg/day (5 mg administered orally twice per day) showed a significantly increased M value with a positive change from baseline with respect to placebo (p=0.002, Mann-Whitney) and pooled patients treated with 10 mg/day or 20 mg/day (10 mg administered orally twice per day) showed a highly significant positive change from baseline M values when compared to placebos (p=0.0007, Mann-Whitney). For patients having essentially normal glucose utilization (M>5), there was no statistically significant difference in a change in M value between placebo treated patients and patients treated with 10 mg/day (n=5), 20 mg/day (n=5) or pooled treated patients (n=10). In the overall protocol with no consideration for M value subgroups, the pooled patients had a significantly increased M value (p=0.0109, Mann-Whitney), as well as the 10 mg/day group (p=0.04, Mann-Whitney) and the 20 mg/day group (p=0.04, Mann-Whitney).

### Example 6

The Phase II, double-blinded placebo controlled 12-week dosing trial of example 4 enrolled 96 subjects with a hemoglobin A1c (HbA1c) level in excess of 7.5 percent and were being treated with a stable metformin dose as described above. The primary objectives of this trial were to evaluate the safety and tolerance of 17α-ethynylandrost-5-ene-3β,7β,17β-triol, 10 mg per day (5 mg twice daily), compared to placebo from baseline to week 12 and to evaluate the change in HbA1c from baseline to week 12 in the 17α-ethynylandrost-5-ene-3β,7β,17β-triol treated group when compared to the placebo group.

An analysis of activity (HbA1c) was performed on all subjects that completed dosing on day 84 of the study (72 patients of the total 96 that enrolled). In the analysis on the entire 72 patient population, there was no statistical difference between treatment and placebo for HbA1c.

A retrospective analysis of data was performed on the subpopulation of patients that represented an obese, insulin-resistant, diabetic population. This analysis included patients who met the following criteria at baseline: BMI greater than or equal to 28; fasting plasma insulin levels greater than or equal to 4 μU/mL; fasting plasma C-peptide levels greater than or equal to 2 ng/mL; and serum monocyte chemoattractant protein-1 (MCP1) levels greater than or equal to 400 pg/mL. This phenotype represented 35% of all 89 patients with values reported for these parameters at baseline. Twenty-two individuals with this phenotype completed the 84 days of dosing. Those treated with 17α-ethynylandrost-5-ene-3β,7β,17β-triol (10 patients) were found to show improvements in clinical parameters compared to placebo patients (12 patients). These included significant trends for a decrease in HbA1c (−0.53%, p=0.06, Mann-Whitney) and a decrease in fasting plasma glucose (−28.75 mg/dL, p=0.09), as well as non-significant decreases in fasting plasma C-peptide (−0.43 ng/mL), fasting plasma insulin (−0.48 μU/mL), fructosamine (−25.75 μmol/L), HOMA2 insulin resistance (−0.65 IR), and increases in HOMA2 insulin sensitivity (11.3% S) and HOMA2 beta cell function (17.95% B). These changes in secondary indicators of activity were consistent with the observed decreases in HbA1c and glucose in this diabetic subpopulation. Sensitivity analysis using last observation carried forward indicated that individuals in this subpopulation who completed at least 29 days of dosing showed improvement.

### Example 7

Treatment of diabetes chemotherapy-naïve responder subpopulation. Based on the results described in Example 6 above, a Phase II, double blind, randomized, placebo-controlled study of the drug 17α-ethynylandrost-5-ene-3β,7β,17β-triol when administered orally for 12 weeks to adult patients with Type 2 Diabetes Mellitus (T2DM) was designed and initiated. The drug was formulated in white gelatin capsules, each of which contained 5 mg of drug in excipients consisting of sodium lauryl sulfate, microcrystalline cellulose, crospovidone, and magnesium stearate. The placebo was provided as identical capsules containing the same excipients as the drug capsules, except lacking the drug. For this protocol, drug-naïve patients were defined as individuals diagnosed as type 2 diabetic patients who (i) had never received drug treatment for T2DM, (ii) had not received therapy with rosiglitazone, pioglitazone, exendin-4 or sitagliptin, alone or in combination within 6 months prior to screening or (iii) had not received another antidiabetic drug therapy during the 3 months prior to screening.

This protocol required a subpopulation of patients 18-65 years of age, inclusive, who met the following criteria at baseline: a body mass index greater than or equal to 28 (at least 28 kg/m² but no more than 37 kg/m² for females and no more than 39 kg/m² for males); fasting plasma insulin levels greater than or equal to 4 μU/mL; fasting plasma C-peptide levels greater than or equal to 2 ng/mL; and serum MCP1 levels greater than or equal to 400 pg/mL. Other inclusion criteria at screening were (i) a fasting blood glucose level of 255 mg/dL; (ii) HbA1c levels of 7.0-10.5%; and (iii) stable weight (±5%) for 3 months prior to screening. The duration of dosing for this protocol is 12 weeks with later follow up observations. After screening and enrollment, eligible consenting patients were dosed with 10 mg/day of 17α-ethynylandrost-5-ene-3β,7β,17β-triol (5 mg b.i.d.). The study is ongoing. Conduct of this protocol allows (i) evaluation of the changes in HbA1c from baseline to week 16 in the 17α-ethynylandrost-5-ene-3β,7β,17β-triol (drug) treated group compared to the placebo group, (ii) evaluation of the safety and tolerance of the drug compared to placebo from baseline to week 12, and (iii) evaluation of the effect of the drug on fasting blood glucose, lipids (cholesterol, HDL, LDL, TG, etc.) or insulin sensitivity (insulin, C-peptide, HOMA2, fructosamine, etc.) over time.

### Example 8

Blood levels of 17α-ethynylandrost-5-ene-3β,7β,17β-triol were determined in adult patients dosed with a range of dosages as oral immediate release formulations. The formulations were essentially as described in example 3, with capsules containing 1 mg/capsule, 5 mg/capsule, 10 mg/capsule, 25 mg/capsule or 50 mg/capsule. These unit doses were filled into hard gelatin capsules (size #1 for 10, 25 and 100 mg doses; size #2 for the 1 and 5 mg doses) and micronized compound (D₉₀˜8-10 pm) was used.

| | mg/ | mg/ |
|---|---|---|
| | capsule | capsule |
| 17α-ethynylandrost-5-ene-3β,7β,17β-triol | 25 | 100 |
| Sodium lauryl sulfate, USP | 50 | 50 |
| Microcrystalline cellulose, USP (Avicel PH 102) | 108 | 61 |
| Crospovidone, USP (Polyplasdone XL-10) | 65 | 37 |
| Magnesium stearate, USP | 2 | 2 |

| | mg/ | mg/ |
|---|---|---|
| | capsule | capsule |
| 17α-ethynylandrost-5-ene-3β,7β,17β-triol | 5 | 10 |
| Sodium lauryl sulfate, NF | 25 | 50 |
| Microcrystalline cellulose, USP (Avicel PH 102) | 74 | 117 |
| Crospovidone, USP (Polyplasdone XL-10) | 45 | 71 |
| Magnesium stearate, USP | 1 | 2 |

| | mg/capsule |
|---|---|
| 17α-ethynylandrost-5-ene-3β,7β,17β-triol | 1 |
| Sodium lauryl sulfate, NF | 5 |
| Microcrystalline cellulose, USP (Avicel PH 102) | 98 |
| Crospovidone, USP (Polyplasdone XL-10) | 45 |
| Magnesium stearate, USP | 1 |

Two capsules containing 1 mg of 17α-ethynylandrost-5-ene-3β,7β,17β-triol were administered twice per day to patients receiving a 4 mg/day total dose. In patients (n=2) dosed with 2 mg twice per day peak blood levels were about 1-2 ng/mL, which were observed at about 2-3 hours after dosing. Blood levels of the compound were at or above about 0.5 ng/mL for over 2 hours/day. In these patients, pre-diabetics with hyperglycemia, one patient showed improved glucose control as seen by an increased M value after 28 days of dosing 4 mg/day. In other patients receiving 4 mg/day for 28 days, there was a lower degree of response than patients receiving higher doses and 4 mg/day was considered to be the minimal effective dose.

Drug blood levels in patients (n=2) given a 50 mg dose of 17α-ethynylandrost-5-ene-3β,7β,17β-triol twice per day (100 mg/day) had peak serum levels of about 80-150 ng/mL and serum levels remained at or above 10 ng/mL for the entire day. These results indicated that oral dosing of 17α-ethynylandrost-5-ene-3β,7β,17β-triol at levels needed to attain serum levels to a maximum of about 200 ng/mL would be practical and safe in adults based on safety studies of the drug in dogs. Collectively, the data from patients dosed with 4 mg/day to 100 mg/day indicated that demonstrable efficacy and acceptable safety for disease treatments could be attained within this range of doses, which would translate to blood drug levels of about 0.5 ng/mL to about 200 ng/mL for about 2 hours to at least about 4 hours per day.

Oral dosing of 17α-ethynylandrost-5-ene-3β,7β,17β-triol to attain blood levels significantly above about 200 ng/mL was considered to be impractical because that would require needlessly large doses without a commensurate increase in therapeutic benefit and/or a potential effect on liver enzymes, i.e., induction of cytochromes that could occur (but was not observed at the 100 mg/day dose level) if drug levels increased over time from, e.g., dose building or prolonged levels of drug above about 50-75 ng/mL each day. Variation in individual patients or their status was observed. For example, the Cmax for 17α-ethynylandrost-5-ene-3β,7β,17β-triol varied depending on fasted or fed status, with fasted Cₘₐₓ levels being lower (up to about 60%) compared to patients dosed after a meal. Total drug exposure levels were generally somewhat higher in adult females than adult males and Cₘₐₓ levels in females were also sometimes higher. However, the ranges of serum levels that were observed over time were within the range of 0.5 ng/mL to about 200 ng/mL for about 2 hours to at least about 4 hours per day. Other data, summarized below, indicated that blood drug levels of about 3 ng/mL to about 50 ng/mL for about 2 hours to at least about 4 hours per day (preferably about 6 hours to at least about 12 hours) was optimal for eliciting therapeutic benefit while still minimizing or avoiding toxicity in most treated individuals, particularly in responder patients having a BMI of about 28.

Drug blood levels in patients (n=2) given a 10 mg dose of 17α-ethynylandrost-5-ene-3β,7β,17β-triol twice per day (20 mg/day on a single day) had peak serum levels of about 10-30 ng/mL and serum levels remained at or above about 3 ng/mL for the entire day. Drug blood levels in patients (n=2) given a 25 mg dose of 17α-ethynylandrost-5-ene-3β,7β,17β-triol twice per day (50 mg/day on a single day) had peak serum levels of about 100-50 ng/mL and serum levels remained at or above about 3 ng/mL for the entire day. These results indicated that oral dosing of 17α-ethynylandrost-5-ene-3β,7β,17β-triol at levels needed to attain serum levels to a maximum of about 200 ng/mL would be practical and safe in adults based on safety studies of the drug in dogs. Collectively, the data from patients dosed with 4 mg/day or 100 mg/day indicated that demonstrable efficacy and acceptable safety for disease treatments could be attained within this range of doses, which would translate to blood drug levels of about 0.5 ng/mL to about 200 ng/mL for about 2 hours to at least about 4 hours per day.

Analysis of some of the evaluable patients from example 1 gave the following results. The mean Cₘₐₓ in pre-diabetic hyperglycemic females (n=6) receiving 10 mg of 17α-ethynylandrost-5-ene-3β,7β,17β-triol per day (5 mg twice per day) was 11.2 ng/mL and 3.8 in males (n=2). The mean Cₘₐₓ in females receiving 20 mg/day (10 mg twice per day) (n=3) was 13.8 ng/mL and 15.1 in males (n=4). From a clinical trial in rheumatoid arthritis patients (not patients from example 1), the mean Cₘₐₓ in females receiving 40 mg/day (20 mg twice per day) (n=6) was 27.4 ng/mL and 43.7 in males (n=2). Collectively, this data indicated that blood drug levels of about 3 ng/mL to about 50 ng/mL for about 2 hours to at least about 4 hours per day (or about 6 hours to at least about 12 hours) was obtained by these doses, which were well within current known safety limits. Drug exposure over 24 hours (AUC₀₋₂₄) in patients receiving 10 mg/day (5 mg twice per day) ranged from 15.7 ng·hr/mL to 190 ng·hr/mL and from 72 ng·hr/mL to 198 ng·hr/mL in patients receiving 20 mg/day (10 mg twice per day). Drug exposure in patients receiving 40 mg/day (20 mg twice per day) ranged from 60 ng·hr/mL to 209 ng·hr/mL. Collectively, this indicates that 17α-ethynylandrost-5-ene-3β,7β,17β-triol drug exposure levels (AUC₀₋₂₄) of about 15 ng·hr/mL to about 220 ng·hr/mL, preferably about 50 ng·hr/mL to about 200 ng·hr/mL, corresponds to optimal dosing in terms of maximal safety and efficacy.

### Example 9

Treatment of spontaneous autoimmune diabetes in non-obese diabetic (NOD) mouse model. The NOD model is used as an animal model for human type 1 diabetes. The NOD mouse strain spontaneously develops autoimmune (type 1) diabetes by a process that has similarities to human type 1 diabetes mellitus, e.g., immune-mediated selective destruction of the pancreatic islet 13-cells that produce insulin (M. A. Atkinson and N. K. Maclaren, New England J. Medicine, 331:1428-1436 1994). There is a higher incidence of diabetes (i.e., hyperglycemia, glucosuria, and hypoinsulinemia) in female vs. male NOD mice (80-90% vs. 10-20%, respectively). Disease incidence in this animal model is first observed at approximately 12 to 15 weeks of age with maximum incidence usually peaking 5 to 6 weeks later.

Methods. An oral formulation containing 17α-ethynylandrost-5-ene-3β,7β,17β-triol in 30% w/v β-cyclodextrin sulfobutyl ether sodium salt (Captisol®; CyDex, Inc., Overland Park, Kans.) was used. Six-week old female NOD mice (approximately 20-25 grams) were purchased from The Jackson Laboratories (Bar Harbor, Me.) and housed in a vivarium under pathogen-free conditions. The animals were acclimated for at least one week prior to initiation of oral dosing with 17α-ethynylandrost-5-ene-3β,7β,17β-triol. Animals were randomized into 5 per cage upon receipt and cages were subsequently randomized into dosing groups.

Female NOD mice were treated once daily with vehicle, 17α-ethynylandrost-5-ene-3β,7β,17β-triol (20 or 80 mg/kg in 100 μL, p.o.), or dexamethasone (0.25 mg/kg, 100 μL, i.p.) from 15 to 25 weeks of age (i.e., before the first incidence of diabetes; at the University of Catania), from 13 to 19 weeks of age (2 days after the first incidence of diabetes), or from 12 to 26 weeks of age (9 days after the first incidence of diabetes). Blood glucose was monitored using glucometers (ENCORE Glucometer, Bayer Corp., Elkhart, Ind.) at weekly intervals, beginning at 10 weeks of age. Mice with blood glucose levels 200 mg/dL on two consecutive occasions were considered diabetic. The percentage of animals with diabetes over the course of the experiment was monitored.

Histological assessment of insulitis and functional 13-cell content was assessed at the time of disease diagnosis or at the end of the 26-week study (i.e., disease-free mice). Pancreata (after CO₂ euthanasia) were immediately isolated and placed in 10% buffered formalin at room temperature overnight, and then embedded in paraffin. After de-paraffinizing, pancreas samples were rehydrated and 5 μm sections were prepared and fixed on glass slides. Some sections were stained with hematoxylin and eosin (H&E) while others were stained with anti-insulin antibody as follows; prepared sections were immersed in Tris-Buffered Saline (TBS: 10 mM Tris buffer, pH 7.4, 0.15 M NaCl) containing 1% of bovine serum albumin (BSA) and 5% normal goat serum for 2 h at room temperature followed by incubation with 2 μg/mL rat anti-insulin IgG2a monoclonal antibody (MAB1417; R&D Systems, Minneapolis, Minn.) in TBS/1% BSA overnight at 4° C. in a humidified chamber. After washing with TBS/0.5 Tween-20, sections were covered with biotinylated anti-rat IgG secondary antibody (BD biosciences, 1:1,000) for 1 h at room temperature, followed by washing with TBS/Tween-20 and incubation with streptavidin-horseradish peroxidase for 30 min. Sections were then incubated with chromogen substrate, DAB (BD Biosciences), for 5 min and counterstained with hematoxylin. The degree of insulitis (leukocyte infiltration per islet; H&E) and functional 13-cell content per islet (insulin production) was scored using a scale of 0 to 3 in which 0=none, 1=few, 2=moderate, 3=high content of inflammatory cells or functional β-cells. Pancreata that were scored were obtained from a randomly-selected portion of mice from each cohort and scores were reported as the mean±SEM for each cohort.

Results. The first incidence of diabetes observed on week 19 was 25%, 8%, and 0% in the vehicle, 17α-ethynylandrost-5-ene-3β,7β,17β-triol, and dexamethasone treated cohorts, respectively. The maximum disease incidence during treatment was observed from weeks 23 through 25 in which the vehicle-treated cohort was 66% and that of the 17α-ethynylandrost-5-ene-3β,7β,17β-triol and dexamethasone treated cohorts was significantly lower, 25% and 8%, respectively (p<0.05; Chi-Squared test). Disease incidence slightly increased in all groups after treatment stopped (week 26 through 31). The mean body weights of each cohort at the end of the study were not significantly different (at week 31, the mean±SEM body weights were 24.3±1.5 g, 24.3±0.6 g, 24.0±0.8 g, for the vehicle, 17α-ethynylandrost-5-ene-3β,7β,17β-triol, and dexamethasone treated cohorts, respectively).

Two additional studies were performed in which single daily oral doses of 17α-ethynylandrost-5-ene-3β,7β,17β-triol were administered either 2 days or 9 days after the first incidence of diabetes. In each case, an 80 mg/kg dose of 17α-ethynylandrost-5-ene-3β,7β,17β-triol suppressed the progressive increase in disease incidence that was observed in the vehicle treated cohorts. There was a significantly different response (p=0.009, Fischer's Exact test) between 20 and 80 mg/kg of 17α-ethynylandrost-5-ene-3β,7β,17β-triol, where 20 mg/kg administration was ineffective. The mean±SEM body weights of each cohort at the end of this study (i.e., Week 25) were 21.0±0.5 g, 24.7±0.3 g, 24.6±0.8 g, and 21.3±0.3 g, for the vehicle, 80 mg/kg 17α-ethynylandrost-5-ene-3β,7β,17β-triol, 20 mg/kg 17α-ethynylandrost-5-ene-3β,7β,17β-triol, and dexamethasone treated cohorts, respectively. A meta-analysis was performed across all studies showed that the 80 mg/kg dose led to a significant decrease in disease incidence relative to that of vehicle administration (p=0.007, Fischer's Exact test). These results showed that daily oral treatment with 17α-ethynylandrost-5-ene-3β,7β,17β-triol administered relatively late during the destructive autoimmune process consistently led to a significant suppression of disease incidence in this NOD model of T1 DM.

Treatment with 17α-ethynylandrost-5-ene-3β,7β,17β-triol (80 mg/kg) led to reduced insulitis and was associated with a significant preservation of islet numbers per pancreas and increased functional 13-cell numbers. A significant reduction in the degree of inflammatory leukocyte infiltrate of islets was also observed on histopathologic observation. The results are shown below.

| | | | No. Islets/ | | Insulitis | | β-Cell | |
|---|---|---|---|---|---|---|---|---|
| | Disease | | pancreas | | Score/Islet | | Score/Islet | |
| | Incidence | (N) | (H&E) | (N) | (H&E) | (N) | (Insulin) | (N) |
| vehicle | 0.63 ± 0.18 | (8) | 2.4 ± 0.6 | (8) | 2.7 ± 0.3 | (6) | 1.0 ± 0.3 | (6) |
| drug (20 mg/kg) | 0.78 ± 0.15 | (9) | 2.4 ± 0.9 | (9) | 2.5 ± 0.3 | (6) | 1.5 ± 0.4 | (6) |
| drug (80 mg/kg) | 0.17 ± 0.17* | (6) | 8.0 ± 3.1* | (6) | 0.8 ± 0.4* | (5) | 2.6 ± 0.4* | (6) |
| dexamethasone | 0.20 ± 0.20 | (5) | 8.2 ± 6.3 | (5) | 2.0 ± 0.6 | (3) | 2.3 ± 0.3* | (3) |

*Significantly different from vehicle (Student's t test)

Collectively, the results indicated that 17α-ethynylandrost-5-ene-3β,7β,17β-triol treatment would be effective in treating type 1 diabetes, e.g., according to the methods, embodiments or claims disclosed herein. Such treatments would be beneficial even in relatively late stage disease.

### Example 10

Effects of 17α-ethynylandrost-5-ene-3β,7β,17β-triol treatment on liver and lipid profiles. Treatment of animals with 17α-ethynylandrost-5-ene-3β,7β,17β-triol was shown to inhibit macrophage inflammatory pathways and decrease macrophage chemotaxis in vitro and in vivo. Treatment with the compound was associated with decreased systemic inflammation and decreased hyperglycemia, presumably by lowering gluconeogenic substrates. Increased whole body insulin sensitivity in the animals was also observed. In addition, 17α-ethynylandrost-5-ene-3β,7β,17β-triol treatment modulated lipid metabolism in the liver and decreased circulating cholesterol levels were observed.

Methods. LPS and recombinant TNFα were obtained from Sigma. Anti-SREBP2 was obtained from Abcam (Cambridge, Mass.). Other primary antibodies were purchased from Cell Signaling Technology (Danvers, Mass.) unless otherwise indicated. Murine primary macrophages were elicited by intraperitoneal injection of thioglycollate (3 mL/mouse) in C57BL/6J mice. Macrophages were obtained from intraperitoneal lavage and washed twice. Cells were cultured in RPMI supplemented with 10% fetal bovine serum (FBS) for 3 days and then starved in RPMI supplemented with 0.5% FBS for overnight before the treatment. RAW 264.7 cells and 3T3-L1 cells were cultured essentially as described elsewhere (T. Yoshizaki, et al. Molecular and Cellular Biology, 29:1363-1374, 2009).

The study was staggered into 6 cohorts conducted on different days. Forty-two male Zucker Diabetic Fatty (ZDF) rats and six Zucker Fatty rats at 7-week of age were obtained from Charles River laboratories (Wilmington, Mass.). The rats were housed individually in polycarbonate cages in a temperature-controlled room on a 12 h: 12 h light: dark cycle with the lights on at 0600 h. Rats were fed ad libitum except during the experiments. After one week of acclimation to environmental conditions, the ZDF rats began daily oral treatment for 32 to 35 days with vehicle (n=18), 100 mg/kg/day 17α-ethynylandrost-5-ene-3β,7β,17β-triol (n=15), or 10 mg/kg/day rosiglitazone (n=9).

Glucose levels were measured using a One-Touch glucometer (Lifescan). Extra serum samples were collected for free fatty acid (FFA), free glycerol, pyruvate, and lactate measurements. Glucose, insulin and pyruvate tolerance tests were performed on 6 hr fasted rats. For GTT, animals were orally gavaged with glucose (1 g/kg), whereas for ITT, 0.35 units/kg insulin (Novolin R, Novo-Nordisk) was injected intraperitoneally. Blood samples were taken at basal and 15, 30, 60, 90 and 120 min after injection. For the pyruvate tolerance test (PTT), 1 g/kg of pyruvate was used.

Rat hyperinsulinemic euglycemic clamp studies were performed as previously described (H. Satoh et al. Diabetes 54:1304-1313, 2005) with modifications. Dual jugular venous cannulae and one carotid arterial cannula were implanted in rats. The rats were allowed to recover for 4 to 5 days before clamp procedure. The hyperinsulinemic euglycemic clamp experiments began with a priming injection (7.5 μCi/0.2 ml) and constant infusion (0.25 μCi/min) of D-[3-³H] glucose (Du Pont-NEN, Boston, Mass.). After 60 min of tracer equilibration and basal sampling at t=−10 and 0 min, glucose (50% dextrose, variable infusion; Abbott) and tracer (0.25 μCi/min) plus insulin (20 μU/kg/min) were infused into the jugular vein. Small blood samples were drawn at 10-min intervals and immediately analyzed for glucose to maintain the integrity of the glucose clamp throughout the duration of the experiment. Blood samples were taken at t=−60 (start of experiment), −10, 0 (basal), 110, and 120 (end of experiment) min to determine glucose-specific activity and insulin, and free fatty acid (FFA). To ensure accuracy, basal and terminal sampling was performed twice at 10-min intervals. The achievement of steady-state conditions (100 mg/dl±5 mg/dl) was confirmed at the end of the clamp by measuring blood glucose every 10 min and ensuring that steady state for glucose infusion and plasma glucose levels were maintained for a minimum of 20 min. All blood samples were immediately centrifuged, and plasma was stored at −80° C. for subsequent analysis.

Following a 3-day recovery after clamp, rats were fasted 6 hours and euthanized. Tissues were harvested at basal state or acute insulin-stimulated state (5 U/kg). Insulin levels were analyzed by ELISA assay (Alpco Diagnostics). FFA levels were measured by enzymatic assay (Wako Diagnostics). Glycerol levels were measured using an assay kit from Sigma. Lactate and pyruvate assay kits were obtained from Biovision. TNFα and IL-1βwere measured by ELISA assays (Biosource).

Immunohistochemistry and quantification of crown-like structure (CLS) were done. These studies were conducted using paraffin-embedded adipose (epididymal) tissue sections from the rats incubated with MAC-2 antibody (Abcam) at a 1:100 dilution overnight at 4° C. Subsequently, biotinylated anti-rat secondary antibody (BD Bioscience) was used at 1:100 dilution, followed by 1:500 HRP-Streptavidin (Jackson ImmunoResearch) and development in substrate chromogen. Slides were counterstained with Mayer's and mounted with Vectashield mounting media with DAPI (Vector). Both bright field (MAC-2) and fluorescent photographs (DAPI) were taken of 3 representative fields per slide in a blinded fashion using a fluorescent microscope (10× objective).

Gene expression analyses used total RNA extracted from tissue with Purelink total RNA purification system (Invitrogen) following the manufacturer's protocol. Total RNA (2 μg) was reverse-transcribed using iScript cDNA Synthesis Kit (Bio-Rad Laboratories, Hercules, Calif.). Primer sequences used in the PCR reactions were chosen based on the sequences available in GenBank. Primer sequences are available upon request. PCR was carried out using iTaq SYBR Green supermix (Bio-Rad Laboratories, Hercules, Calif.) on an MJ Research Chromo4 Real Time PCR system (Bio-Rad Laboratories BV, Hercules, Calif.). The mRNA expression of all genes reported is normalized to multiple housekeeping genes (34B4, RNA polymerase II, and cyclophilin A) and comparable results were found. For Quantitative Nuclease Protection Assay (qNPA), cells were lysed in lysis buffer after various treatments. The qNPA ArrayPlate assays were performed by High Throughput Genomics, Inc.

The macrophage chemotaxis assay was conducted with differentiated 3T3-L1 adipocytes (day +11 post differentiation) incubated for 24 hours with compounds (10 ng/mL TNFα with or without 100 nM 17α-ethynylandrost-5-ene-3β,7β,17β-triol) or vehicle in DMEM with 0.2% FFA- and endotoxin-free BSA. Conditioned medium (CM) was collected and kept frozen in single-use aliquots. For the chemotaxis assay, 600 μl/well of adipocyte CM was aliquoted into 24-well tissue culture plates. 2×10⁵ RAW264.7 macrophage cells resuspended in DMEM containing 0.2% BSA were plated in the upper transwell chamber (8 pm, 24-transwell format; Corning, Lowell, Mass.). After 3 hours of migration, the RAW264.7 cells were fixed in formalin and stained with DAPI. Cells in the upper chamber that had not migrated were removed. Cells found on the filter facing the lower chamber were counted as cells having performed chemotaxis and quantified with the Simple PCI imaging software (Compix Inc., Cranberry Township, Pa.).

For Western blots, cells were serum starved and pretreated with DMSO or 17α-ethynylandrost-5-ene-3β,7β,17β-triol (100 nM) for overnight before LPS (100 ng/ml) or TNFα (10 ng/ml) stimulation for the indicated times, and then were lysed in radioimmunoprecipitation buffer containing protease inhibitor cocktail (Roche) and phosphatase inhibitor cocktail (Sigma). Total cell lysates were separated by 10% SDS-PAGE. The blots were incubated first with blocking buffer (Tris-buffered saline containing 0.05% Tween 20 and 5.5% nonfat milk) followed by primary antibodies in blocking buffer or Tris-buffered saline containing 2% BSA. The bound antibodies were detected by horseradish peroxidase-conjugated secondary antibodies (1:4000, v/v) in blocking buffer and visualized by enhanced chemiluminescence.

Unless otherwise noted, data were analyzed by ANOVA followed by Tukey post hoc tests. Individual pair-wise comparisons were performed using two-tailed t test. Analysis was performed using Excel (Microsoft, Redmond, Wash.) or Prizm (GraphPad Software, Inc., San Diego, Calif.).

Results. Treatment with the toll-like receptor 4 (TLR4) ligand lipopolysaccharide (LPS) was used to activate proinflammatory signaling cascades, including phosphorylation of IKK and mitogen-activated protein (MAP) kinases, such as JNK, p38, and extracellular signal-regulated kinases (ERK). As a result, various inflammatory genes were up-regulated as determined by a quantitative nuclease protection assay (pNPA) array, e.g., TNFα, IL-6). Pretreatment with 17α-ethynylandrost-5-ene-3β,7β,17β-triol partially, but significantly, blocked the activation of IKK, JNK, p38 and ERK (p<0.05). Although IκB phosphorylation and degradation were not influenced by 17α-ethynylandrost-5-ene-3β,7β,17β-triol treatment, NFκB phosphorylation was reduced. Concordantly, LPS-induced transcription of IL-1β, IL-6, IL-12, TNFα, Cxcl1, Nos2, Cxcl10, and Cxcl1 was significantly reduced. These results suggest that 17α-ethynylandrost-5-ene-3β,7β,17β-triol ameliorated intracellular inflammatory responses elicited by TLR4 signaling.

Conditioned media (CM) from 3T3-L1 adipocytes was used to induce chemotaxis of RAW 264.7 monocyte/macrophages. CM from TNFα-treated adipocytes markedly stimulated macrophage migration, which was reduced by ˜30% (p<0.05) when adipocytes were pretreated with 17α-ethynylandrost-5-ene-3β,7β,17β-triol. In addition, macrophage secretion of inflammatory cytokines, such as monocyte chemotactic protein-1 (MCP-1/CCL2) and chemokine (C-C motif) ligand 5 (CCL5/RANTES), was augmented by TNFα-treated adipocyte CM, but that was significantly decreased by pretreatment of the adipocytes with 17α-ethynylandrost-5-ene-3β,7β,17β-triol (p<0.05).

Zucker diabetic fatty (ZDF) rats were treated with placebo vehicle, 17α-ethynylandrost-5-ene-3β,7β,17β-triol (100 mg/kg/d), or rosiglitazone (10 mg/kg/d) for 4 weeks. The ZDF rat is a model of obesity, insulin resistance, and diabetes and is develops hyperinsulinemia at 8 to 9 weeks of age and hyperglycemia after 9 to 10 weeks of age. Treatments were initiated at 8 weeks of age and progression of diabetes in the animals was monitored. Treatment with 17α-ethynylandrost-5-ene-3β,7β,17β-triol completely normalized fasting and fed glucose levels throughout the study. Thus, one week of treatment with 17α-ethynylandrost-5-ene-3β,7β,17β-triol was sufficient to normalize fasting and fed glucose levels, as well as plasma insulin levels. Rosiglitazone, an insulin sensitizer, was used as a positive control and it ameliorated hyperglycemia and hyperinsulinemia as expected.

Euglycemic-hyperinsulinemic clamp studies were used to measure in vivo insulin sensitivity. Both 17α-ethynylandrost-5-ene-3β,7β,17β-triol and rosiglitazone treatment led to an increase in the glucose infusion rate and insulin-stimulated glucose disposal, with rosiglitazone being more robust. Basal rates of hepatic glucose production (HGP) were equally reduced by 17α-ethynylandrost-5-ene-3β,7β,17β-triol and rosiglitazone treatment. Because basal HGP is the major contributor to basal hyperglycemia, this result was consistent with the marked reduction in basal glucose levels that were observed in these animals.

In ZDF rats, development of diabetes mellitus is accompanied by functional and morphological kidney damage that resembles human diabetic nephropathy. Kidneys from the treated rats were dissected and the gross pathology of the kidneys due to diabetes was found to be greatly ameliorated by treatment with 17α-ethynylandrost-5-ene-3β,7β,17β-triol. Collectively, the results show that the 17α-ethynylandrost-5-ene-3β,7β,17β-triol can prevent or delay the onset of diabetes and its complications.

Increased adipose tissue macrophage (ATM) content is an important component of the chronic tissue inflammatory state in obesity. To determine the effect of 17α-ethynylandrost-5-ene-3β,7β,17β-triol on macrophage-mediated inflammation in vivo, histological analysis of adipose tissue sections from control and treated rats was performed. There was a marked reduction in ATM content, as measured by staining for the macrophage specific marker Mac-2 in the treated rats. This directly demonstrates decreased ATM accumulation. There also was a decrease in a variety of inflammatory markers, including TNFα and MCP-1, in adipose tissue from the 17α-ethynylandrost-5-ene-3β,7β,17β-triol (p<0.05) and rosiglitazone treated rats (p<0.05).

Dyslipidemia is usually present in patients with insulin resistance or diabetes. Increased tissue lipid accumulation has been demonstrated in several animal models of obesity and diabetes, including ZDF rats. Lipid profiles in three key insulin-responsive tissues: liver, fat, and skeletal muscle were assessed. Treatment with 17α-ethynylandrost-5-ene-3β,7β,17β-triol led to a marked decrease in intracellular triacylglycerol (TAG) content in livers as did rosiglitazone. Treatment with 17α-ethynylandrost-5-ene-3β,7β,17β-triol significantly reduced hepatic cholesteryl ester levels by 73%, with total cholesterol content in the liver reduced by 15%. Gene expression measurements demonstrated increased low density lipoprotein receptor (LDLR) and HMG CoA reductase expression with 17α-ethynylandrost-5-ene-3β,7β,17β-triol treatment. Treatment also led to a reduction in serum cholesteryl esters (59% decrease) and total cholesterol levels (25% decrease), despite an increase in serum free cholesterol levels. The opposite was true for rosiglitazone treatment. In epididymal white adipose tissue (WAT), 17α-ethynylandrost-5-ene-3β,7β,17β-triol treatment reduced the level of free cholesterol. Other major lipid classes were not significantly altered by. Diacylglycerol and fatty acid contents were elevated as expected by rosiglitazone treatment, which is known to enhance the glycerolipid turn over rate in white adipose tissue.

### Example 11

### 17α-Ethynylandrost-5-ene-3β,7β,17β-triol was prepared as follows

Synthesis of 3β,7β-bis-(trimethylsiloxy)-5-androsten-17-one: A mixture of 14.87 Kg of androst-5-en-17β-one-3β,7β-diol, 23.8 Kg 1,1,1,3,3,3-hexamethyldisilazane (HMDS) and 0.7 Kg saccharin catalyst in 100 L acetonitrile was heated to reflux for 8 hours with stirring under a nitrogen atmosphere. Liberated ammonia was purged under slight vacuum. The reaction volume was then reduced by distillation to collect 30 L of distillate (about 2 h). The reaction volume was further reduced to half of the original reaction volume by distillation under reduced pressure (700 mmHg), which requires about 2 h of heating at 50° C. The resulting uniform thick slurry was cooled to 5° C. (requires about 3 h), with additional acetonitrile added to maintain a minimum mixing volume, and held at that temperature for 1. The precipitated product was collected by filtration and dried at 45-50° C. under vacuum (29 mmHg) to a loss on drying (LOD) of not more than 1% (requires 20 h) to provide 16 Kg (81% yield) of the title compound (95% purity).

Synthesis of 17α-ethynyl-5-androstene-3β,7β,17β-triol: To 11.02 Kg TMS-acetylene in 56.5 L tetrahydrofuran (THF) at −27° C. under a nitrogen atmosphere was added 8.51 L 10M n-BuLi. The n-butyl lithium was added very slowly to maintain a temperature at −7 to −27° C. (about 2 h) and the resulting reaction was stirred 10 min. at approximately 0° C. to produce TMS-lithium-acetylide. To the TMS-lithium-acetylide solution was added a solution of 25.41 Kg of 313,713-bis-(trimethylsiloxy)-5-androsten-17-one in 95.3 L THF filtered through a 25 μM filter while allowing the reaction temperature to rise to 20-25° C.

After addition was completed, the reaction temperature was increased to 40-45° C. To quench the reactor contents, 31.8 L of methanol was added over a period of about 1 h followed by 3.81 Kg KOH in 18.4 L of water giving a final reactor temperature of 50° C. Liberated acetylene is purged under slight vacuum. The reactor contents were then concentrated by distillation at 80° C. for 1 h then under vacuum (175 mmHg) at about 70° C. (with an initial temperature of 25° C. to avoid bumping) to half of the original pot volume. The residue was cooled to about 10° C. and 35.0 Kg of deionized water was added, followed by 16.4 Kg 12N HCl while maintaining a pot temperature of about 10° C. and giving a final pH of 1. Additional 26.0 kg deionized water was added and the resulting mixture was stirred at about 5° C. for 1 h. The resulting slurry was filtered and washed with 75/25 mixture of methanol/water (16.9 L methanol, 5.6 L water). The collected solids were dried under vacuum (28 in Hg) at 45° C. for 12 h for a loss on drying of no more than 0.5% to provide 9.6 Kg of the title compound (83% yield).

Preparation of 17α-ethynylandrost-5-ene-3β,7β,17β-triol by this method using starting material that is substituted at the 7-position yields a product with essentially no by-products that are unsubstituted at the 7-position, eliminating any need to remove such potential impurities.

### Example 12

A phase 2 clinical treatment protocol was conducted in type 2 diabetes patients using a monotherapy consisting of 17α-ethynylandrost-5-ene-3β,7β,17β-triol. The patients had not been treated with another antidiabetic agent or any insulin sensitizer such as metformin for at least 90 days before the start of dosing. The patients had a BMI of least 28. The patients (n=51) received 10 mg/day of 17α-ethynylandrost-5-ene-3β,7β,17β-triol (5 mg twice per day) for 84 days and were then followed until day 112. Effects of treatment were examined in subsets of patients having differing BMI values. Examination of changes in hemoglobin A1c (HbA1c) showed activity in patients (n=22) with BMI greater than 31.3 kg/m², with a HbA1c decrease of 1.1% observed at day 112, p=0.041, decreased HbA1c at day 84 to <7% (2/9 treated), 0/14 (placebo). Other observed effects in this patient population were a fructosamine decrease of 30 mM at day 112, a trend to decreased C-reactive protein at day 57 (p=0.053) and decreases of >600 pg/mL in serum MCP-1 days 29, 57 and 84. The observed effects were less pronounced in patients having a BMI of less than about 31, although the efficacy of the compound in larger patient groups would be expected to be observed at a BMI range of about 30 and above (obese). These results showed that in treatment naïve patients, 17α-ethynylandrost-5-ene-3β,7β,17β-triol was particularly effective in patients with a BMI of at least about 31.

To the extent not already indicated, it will be understood by those of ordinary skill in the art that any of the various specific embodiments, analysis methods, compounds or compositions described herein may be modified to incorporate other appropriate features, e.g., where one or more protocol steps in a disclosed embodiment is added to or combined with any other compatible protocol step, method or embodiment described herein.

References cited herein are incorporated herein by reference.

What is claimed is:

1. A treatment method for a patient(s) having pre-diabetic hyperglycemia or type 2 diabetes, comprising,
(a) determining the body mass index (BMI) of the patient(s);
(b) in a patient(s) of step (a) that has a BMI greater than 29.9 (is obese), determining the fasting blood or serum glucose level and/or the post prandial glucose level after an oral 75 g glucose load;
(c) selecting patient(s) with a fasting blood glucose level of at least 110 mg/dL or a post prandial glucose level of at least about 140 mg/dL as patient(s) for treatment; and
(d) treating the patient(s) of step (c) with an effective amount of 17α-ethynylandrost-5-ene-3β,7β,17β-triol,
wherein the patient(s) have and wherein the patient(s) has not been treated with metformin and optionally wherein the patient(s) has not been treated with another insulin sensitizer or another antidiabetic agent.

2. The method of claim 1 wherein the patient(s) has a BMI of at least 30.5.

3. The method of claim 1 wherein the patient(s) has a BMI of at least 31.

4. The method of claim 1 wherein, the selected patients of step (c) have (i) a fasting blood glucose level of at least 120 mg/dL or a post prandial glucose level of at least about 150 mg/dL or (ii) a fasting blood glucose level of at least 126 mg/dL or a post prandial glucose level of at least about 200 mg/dL.

5. The method of claim 1 wherein, the patient(s) has
(i) a microvascular disease, wherein the microvascular disease optionally is retinopathy, neuropathy or nephropathy;
(ii) a macrovascular disease, optionally atherosclerosis, arteriosclerosis, a stroke, hypertension or a myocardial infarction;
(iii) a dyslipidemia condition, wherein the dyslipidemia condition is hypercholesterolemia or hypertriglyceridemia; or
(iv) nonalcoholic fatty liver disease or hepatitis wherein the hepatitis optionally is nonalcoholic steatohepatitis, viral hepatitis or acute alcoholic hepatitis.

6. The method of claim 1 wherein the patient(s) has a fasting insulin level of at least 4 μU/mL.

7. The method of claim 4 wherein the patient(s) has a fasting insulin level of at least 4 μU/mL.

8. The method of claim 7 wherein the patient(s) has a BMI of at least 31.

9. A method to treat a patient(s) in need thereof comprising administering an amount of 17α-ethynylandrost-5-ene-3β,7β,17β-triol sufficient to maintain a serum level of about 0.5 ng/mL to about 200 ng/mL for about 2 hours per day to at least about 4 hours per day, wherein the patient(s) has hypercholesterolemia, hypertriglyceridemia, a macrovascular disease, a microvascular disease, nonalcoholic fatty liver disease or hepatitis.

10. The method of claim 9 wherein the amount of 17α-ethynylandrost-5-ene-3β,7β,17β-triol is sufficient to maintain a serum level of about 3 ng/mL to about 50 ng/mL for at least 4 hours.

11. The method of claim 9 wherein about 4 mg to about 400 mg of 17α-ethynylandrost-5-ene-3β,7β,17β-triol in the form of a dose for oral administration that is administered each day as a single dose or twice as two doses each containing about 2 mg per dose to about 200 mg per dose.

12. The method of claim 9 wherein, the patient(s) has
(i) a microvascular disease, wherein the microvascular disease optionally is retinopathy, neuropathy or nephropathy;
(ii) a macrovascular disease, optionally atherosclerosis, arteriosclerosis, a stroke, hypertension or a myocardial infarction;
(iii) hypercholesterolemia or hypertriglyceridemia; or
(iv) nonalcoholic fatty liver disease or hepatitis wherein the hepatitis optionally is nonalcoholic steatohepatitis, viral hepatitis or acute alcoholic hepatitis.
